(19) United States
(12) Patent Application Publication
(10) Pub. No.: US 2005/0009732 A1
(43) Pub. Date: Jan. 13, 2005

(54) Method of treatment and agents useful for same

(75) Inventors: Mathew Vadas, Stirling (AU); Jennifer Gamble, Stirling (AU); Pu Xia, Magill (AU); Lijun Wang, Magill (AU); Olga Sukocheva, Adelaide (AU)

Correspondence Address: MORRISON & FOERSTER LLP, 1650 TYSONS BOULEVARD, SUITE 300, MCLEAN, VA, 22102, US

(73) Assignee: Medvet Science Pty Ltd., Thebarton (AU)

(21) Appl. No.: 10/780,897

(22) Filed: Feb. 19, 2004

Related U.S. Application Data
(63) Continuation-in-part of application No. 10/275,686, filed on Jun. 25, 2003.
(60) Provisional application No. 60/447,707, filed on Feb. 19, 2003.

(30) Foreign Application Priority Data
Feb. 19, 2003 (AU) 2003900729

(51) Int. Cl. A61K 38/17

(52) U.S. Cl. 514/2

(57) ABSTRACT

A method of modulating the growth of a cell, such as a neoplastic or malignant cell from the colon, stomach, lung, brain, bone, esophagus, pancreas, breast, ovary or uterus includes contacting the cell with an agent for a time and under conditions sufficient to modulate the functional activity of sphingosine kinase in which down-regulation of the functional activity of the sphingosine kinase down-regulates growth of the cells and up-regulation of the functional activity of this sphingosine kinase up-regulates the growth of the cell. The down-regulation can reduce the functional activity of this sphingosine kinase to an oncogenic ineffective level. Agents useful in the invention include N,N-dimethylspingosine and DL-threo-dihydrosphingosine

### FIELD OF THE INVENTION

The present invention relates generally to a method of modulating the growth of cells and, more particularly, to a method of down-regulating the growth of neoplastic cells. The present invention is useful, inter alia, in the therapeutic and/or prophylactic treatment of cancers such as, but not limited to, solid cancers such as cancers of the colon, stomach, lung, brain, bone, oesophagus, pancreas, mammary gland (breast), ovary or uterus.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that that prior art forms part of the common general knowledge in Australia.

Sphingosine kinase is a key regulatory enzyme in a variety of cellular responses, Sphingosine-1-phosphate is known to be an important second messenger in signal transduction (Spiegal, S. and Milstein, S. (2002) FEBS Lett, 476:55-57). It is mitogenic in various cell types (Allessenko, A. V., (1998) Biochemistry (Mosc) 63:62-68) and appears to trigger a diverse range of important regulatory pathways including prevention of ceramide-induced apoptosis (Culliver, O., Pirianov, G., Kleuser, B., Vanek, P. G., Coso, O. A., Gutkind, J. S. and Spiegel, S. (1996) Nature 381:800-803), mobilisation of intracellular calcium by an IP₃-independant pathway, stimulation of DNA synthesis, activation of mitogen-activated protein (MAP) kinase pathway, activation of phospholipase D, and regulation of cell motility (for reviews see (Spiegal and Milstein, 2000, supra; Igarashi, Y. (1997) J. Biochem. 122:1080-1087).

Recent studies (Xia, P., Gamble, J. R., Rye, K.-A., Wang, L., Hii, C. S. T., Cockerill, P., Khew-Goodall, Y., Bert, A. G., Barter, P. J., and Vadas, M. A. (1998) Proc. Natl. Acad. Sci. USA 95:14196-14201) have shown that sphingosine-1-phosphate is an obligatory signalling intermediate in the inflammatory response of vascular endothelial cells to tumour necrosis factor-α (TNFα). In spite of its obvious importance, very little is known of the mechanisms that control cellular sphingosine-1-phosphate levels. It is known that sphingosine-1-phosphate levels in the cell are mediated largely by its formation from sphingosine by sphingosine kinase, and to a lesser extent by its degradation by endoplasmic reticulum-associated sphingosine-1-phosphate lyase and sphingosine-1-phosphate phosphatase (Spiegal et al., 1998 supra). Basal levels of sphingosine-1-phosphate in the cell are generally low, but can increase rapidly and transiently when cells are exposed to mitogenic agents. This response appears correlated with an increase in sphingosine kinase activity in the cytosol and can be prevented by addition of the sphingosine kinase inhibitory molecules N,N-dimethylsphingosine and DL-threo-dihydrosphingosine. This indicates that sphingosine kinase is an important molecule responsible for regulating cellular sphingosine-1-phosphate levels. This places sphingosine kinase in a central and obligatory role in mediating the effects attributed to sphingosine-1-phosphate in the cell.

Sphigosine kinase is speculated to play a role in a number of cellular activities including inflammation, calcium mobilisation, cell motility and adhesion molecule expression. However, the precise nature of the cellular activities which are so regulated and the role and mechanistic actions of sphingosine kinase in this regard are only just beginning to be understood. Many of the signals leading to modulation of various cellular activities have not been precisely defined. Elucidating these cellular signalling mechanisms is necessary for the development of therapeutic and prophylactic strategies to disease conditions involving aberrant or otherwise unwanted cellular activities.

In work leading up to the present invention, the inventors have determined that the signalling cascade stimulated by the lipid kinase, sphingosine kinase, plays a major role in oncogenesis and is, in fact, by itself oncogenic when its level of activity is too high. Even in light of preliminary data indicating a role for sphingosine kinase in various cellular activities, the oncogenic activity of sphingosine kinase is a surprising and completely unexpected function attributable to this molecule. Identification of the link between sphingosine kinase and tumour pathogenesis permits the rational design of therapeutic and/or prophylactic regimes for modulating cell growth and, further, the identification of a range of molecules for use in the modulation of cell growth.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides a method of modulating the growth of a neoplastic cell, said method comprising contacting said cell with an effective amount of an agent for a time and under conditions sufficient to modulate the functional activity of sphingosine kinase wherein down-regulation of the functional activity of said sphingosine kinase down-regulates said growth and up-regulation of the functional activity of said sphingosine kinase up-regulates said cell growth.

In another aspect the present invention more preferably provides a method of modulating the growth of a neoplastic cell, said method comprising contacting said cell with an effective amount of an agent for a time and under conditions sufficient to modulate the level of functional activity of sphingosine kinase wherein down-regulation of the functional activity of said sphingosine kinase to an oncogenic ineffective level down-regulates said growth and up-regulation of the functional activity of said sphingosine kinase to an oncogenic effective level up-regulates said cell growth.

In yet another aspect there is provided a method of modulating the proliferation of a neoplastic cell, said method comprising contacting said cell with an effective amount of an agent for a time and under conditions sufficient to modulate the functional activity of sphingosine kinase wherein down-regulation of the functional activity of said sphingosine kinase to an oncogenic ineffective level down-regulates said growth and up-regulation of the functional activity of said sphingosine kinase to an oncogenic effective level up-regulates said cell growth.

In still another aspect there is provided a method of down-regulating the proliferation of a cell, said method comprising contacting said cell with an effective amount of an agent for a time and under conditions sufficient to down-regulate the functional activity of sphingosine kinase to an oncogenic ineffective level.

In still yet another aspect there is provided a method of up-regulating the proliferation of a cell, said method comprising contacting said cell with an effective amount of an agent for a time and under conditions sufficient to up-regulate the functional activity of sphingosine kinase to an oncogenic effective level.

In a further aspect there is provided a method of down-regulating the proliferation of a neoplastic cell, said method comprising contacting said neoplastic cell with an effective amount of an agent for a time and under conditions sufficient to down-regulate the functional activity of sphingosine kinase to an oncogenic ineffective level.

In another further aspect the present invention provides a method of up-regulating the proliferation of a cell, said method comprising contacting said cell with an effective amount of an agent for a time and under conditions sufficient to up-regulate the functional activity of sphingosine kinase to an oncogenic effective level.

In yet another further aspect the present invention contemplates a method for the treatment and/or prophylaxis of a condition characterised by aberrant, unwanted or otherwise inappropriate cell growth in a mammal, said method comprising administering to said mammal an effective amount of an agent for a time and under conditions sufficient to modulate the functional activity of sphingosine kinase wherein down-regulation of the functional activity of said sphingosine kinase down-regulates the subject cell growth and up-regulation of the functional activity of said sphingosine kinase up-regulates the subject cell growth.

In still another further aspect there is provided a method for the treatment and/or prophylaxis of a condition characterised by uncontrolled cell proliferation in a mammal, said method comprising administering to said mammal an effective amount of an agent for a time and under conditions sufficient to down regulate the functional activity of sphingosine kinase to an oncogenic ineffective level.

Another aspect of the present invention relates to the treatment and/or prophylaxis of a neoplastic condition in a mammal, said method comprising administering to said mammal an effective amount of an agent for a time and under conditions sufficient to down-regulate the functional activity of sphingosine kinase to a functionally ineffective level.

In another aspect the present invention relates to the use of an agent capable of modulating the functional activity of sphingosine kinase in the manufacture of a medicament for the modulation of neoplastic cell growth in a mammal wherein down-regulation of the functional activity of said sphingosine kinase down-regulates the subject cell growth and up-regulation of the functional activity of said sphingosine kinase up-regulates the subject cell growth.

Yet another aspect relates to agents for use in modulating the functional activity of sphingosine kinase wherein modulating the functional activity of sphingosine kinase modulates neoplastic cell growth.

In yet another aspect the present invention relates to a pharmaceutical composition comprising a modulatory agent as hereinbefore defied and one or more pharmaceutically acceptable carriers and/or diluents. Said modulatory agents are referred to as the active ingredients.

Yet another aspect of the present invention relates to modulatory agents, as hereinbefore defined, when used in the method of the present invention.

Still yet another aspect of the present invention relates to diagnostic methodology based on screening individuals for the presence of sphingosine kinase or mRNA or protein or the specific forms of sphingosine kinase which are transcribed and/or translated by a given population of cells.

### BRIEF DESCRIPTION OF THE DRAWINGS

FIG. 1 is a graphical representation of the overexpression of SphK in NIH 3T3 cells. (A) Cytosolic SphK activity was measured in the stable transfected cells with pcDNA3-SphK-FLAG (SK-3T3) or empty vector alone (N-3T3). (B) Intracellular S1P levels were measured in [³H]sphingosine-labelled SK-3T3 or N-3T3 cell pools. (C) Proteins from soluble cell lysates were probed with anti-FLAG monoclonal antibodies (M2, Kodak).

FIG. 2 is a graphical representation of SphK overexpression accelerates proliferation in NIH 3T3 cells. (A) Growth curves reveal that SK-3T3 cells (right panel) proliferate more rapid than N-3T3 cells (left panel). Values are the mean of triplicate determinations and similar results were obtained in three independent experiments. (B) Cell growth at saturation density. Cells were counted at absolute confluence (open bars) and 24 h later of confluence (grey bars), respectively. (C) DMS inhibits SphK overexpression induced proliferation. Equal numbers of N-3T3 and SK-3T3 cells were cultured in DMEM containing 10% serum in the presence or absence of DMS (2.5 μM) for 5 days. Media was replaced every day. Data in (B) and (C) are the means±S.D. from three independent experiments done in triplicate.

FIG. 3 is an image of overexpression of SphK, induced NIH 3T3 cell transformation. (A) Focus formation of NIH 3T3 cell transfecants. Cultures transfected with SphK (SK-3T3) or vector alone (N-3T3) were photographed 12 days after transfection (40× magnification). (B) Colony formation in soft agar. N-3T3 and SK-3T3 cells were cultured in growth medium containing 0.33% agar and fed with the medium containing various concentrations of DMS every 2 days. Photographs were taken after 2 weeks of cultures followed by staining with MTT. (C) NIH 3T3 cells were transfected with V12-Ras or v-Src, SphK activity was measured after 48 h transfection. (D) Focus formation assays were performed in V12-Ras, v-Src, or SphK transfected NIH 3T3 cells in the absence or presence of DMS (2.5 μM) over two weeks.

FIG. 4 is an image of cells overexpressing SphK are tumourigenic. (A) Photograph of tumours in the NOD/SCID mice injected with SphK-transfected NIH 3T3 cells. (B) Morphology of a tumour (insert) and the paraffin fixed section stained with hematoxalin and eosin (60× magnification). (C) Whole cell extracts from three individual tumours (lane 4-6) and their peripheral tissues (lane 1-3) and N-3T3 (lane 7) or SK-3T3 cells (lane 8) were analysed by Western blot. The top blot was probed with anti-FLAG antibody and the bottom with anti-actin antibody (Santa Cruz).

FIG. 5 is an image of the overexpression of SK in MCF-7 cells. (a) Immunoblot assay of proteins from lysates of stable transfected MCF-7 cells with overexpression of human wild-type SK1 (SK), a point mutation of SK (SK^{G28D}) or empty vectors. The blot was probed with anti-FLAG monoclonal antibodies (M2). (b) SK activity was measured in the transfected MCF-7 cells treated with or without PMA (100 ng/ml) for 30 min and normalized to total protein levels of each sample. Data are the means±S.E. of three independent experiments.

FIG. 6 is a graphical representation of the effect of SK on E₂-dependent and -independent cell growth. Cell number was calculated and normalized to seeding density from the MTS proliferation assays performed in (a) stable transfected MCF-7 cell lines with overexpression of SK, SK^{G28D} or empty vector incubated in 10% FCS or serum-free media for 5 days; and (b) the transfected cells cultured in 10% FCS media with or without 10 nM E₂ for up to 10 days. Data are the means±S.E. of triplicate determinations and are representative of at least three independent experiments.

FIG. 7 is an image of the effect of SK on E₂-dependent foci formation and colony growth in MCF-7 cells. (a) Foci formation assays were performed in stable transfected MCF-7 cells with overexpression of SK, SK^{G28D} or empty vector. After 10 days exposure to 10 nM E₂ the cultures were photographed at 40× magnification. (b) Colony growth in soft agar was determined in the transfected MCF-7 cells in the absence or presence of 10 nM E₂ for 14 days as described under Methods. Data are the means±S.E. of three independent experiments performed in triplicate.

FIG. 8 is a graphical representation of the effect of E₂ on SK activity in MCF-7 cells. SK activity was measured in MCF-7 cells (a) treated with 10 nM E₂ for the indicated short time course; (b) treated with increasing doses of E₂ for 15 min; (c) treated with 10 nM E₂ for a prolonged time course in the absence or presence of 1 μM actinomycine D (Act D); and (d) treated with 10 nM E₂ for a short time course after pretreated with 1 μM actinomycine D for 4 h or pretreated with 10 nM E₂ for 16 h. Data are the means±S.E. of triplicate determinations and are representative of at least three independent experiments.

FIG. 9 is a graphical representation of the E₂-induced activation of SK being mediated by putative membrane ER. SK activity was measured in (a) MCF-7 and MDA-MB-231 cells treated with 10 nM E₂ for 15 min or 6 h, or without treatment; (b) MCF-7 cells treated with 10 nM E₂, 1 μM E₂-BSA, or pretreated with 50 ng/ml Pertussis toxin (PTX) for 16 h or 10 μM ICI-182,780 (ICI) for 1 h followed by stimulation with 10 nM E₂. Data are the means±S.E. of triplicate determinations and similar results were obtained in three independent experiments.

FIG. 10 is a graphical representation of the effect of SK on E₂-induced increases in [Ca²⁺]ᵢ. Intracellular free Ca²⁺ concentration ([Ca2+]ᵢ) was measured in: (a) stable transfected MCF-7 cells with overexpression of SK, SK^{G82D}, or empty vector stimulated with 1 nM E₂; (b) MCF-7 cells treated with 100 nM S1P or pretreated with 10 μM DMS for 30 min followed by E₂ stimulation; (c) MCF-7 cells treated with 1 μM E₂-BSA or BSA alone; (d) MCF-7 cells treated with 10 μM ICI_{182,780} (ICI) or ICI plus E₂; (e) treated with 100 nM tert-butylhydroquinone (BHQ) or addition of 2 mM EGTA into extracellular media followed by E₂ stimulation; (f) MCF-7 cells treated with E₂ in the absence or presence of 75 μM 2-Aminoethoxydiphenyl borate (2-APB) or DMS (10 μM) plus 2-APB. All calcium tracings shown in the figures are representative of 3˜10 independent experiments.

FIG. 11 is an image of the effect of SK on E₂-induced Erk1/2 phosphorylation. Stable transfected MCF-7 cells with overexpression of SK, SK^{G82D}, or empty vector were pretreated with or without 100 μM PD098059 for 30 min and stimulated with 10 nM E₂ for the indicated time. Cell lysates were subjected to 12% SDS-PAGE and probed with anti-phosphorylated Erk1/2 (p-Erk1/2) (the upper panels) or anti-Erk1/2 (the lower panels) antibodies. Representative blots are shown, and the results were verified in three additional independent experiments.

### DETAILED DESCRIPTION OF THE INVENTION

The present invention is predicated, in part, on the determination of a correlation between cell growth, in particular oncogenesis, and modulation in the level of activity of sphingosine kinase. The identification of this correlation permits the identification and rational design of methodology and products for use in therapy, prophylaxis and diagnosis of disease conditions characterised by aberrant, unwanted or otherwise inappropriate cell growth, in particular, uncontrolled oncogene induced proliferation such as that which occurs in breast cancer.

Accordingly, one aspect of the present invention provides a method of modulating the growth of a neoplastic cell, said method comprising contacting said cell with an effective amount of an agent for a time and under conditions sufficient to modulate the functional activity of sphingosine kinase wherein down-regulation of the functional activity of said sphingosine kinase down-regulates said growth and up-regulation of the functional activity of said sphingosine kinase up-regulates said cell growth

Reference to “sphingosine kinase” should be understood as including reference to all forms of sphingosine kinase or derivative, homologue, analogue, equivalent or mimetic thereof or other molecules having the function of sphingosine kinase and to nucleic acid molecules encoding sphingosine kinase derivative, homologue, analogue, equivalent or mimetic thereof. This includes, for example, all protein or nucleic acid forms of sphingosine kinase or its functional derivative, homologue, analogue, equivalent or mimetic thereof including, for example, any isoforms which arise from alternative splicing of sphingosine kinase mRNA or mutants or polymorphic variants of sphingosine kinase. It should also be understood that reference to a “nucleic acid form of sphingosine kinase” is a reference to a nucleic acid encoding sphingosine kinase and includes reference to any sphingosine kinase regulatory element (such as promoters or enhancers) which regulate the expression of sphingosine kinase and includes regulatory elements which are located at a position other than between the sphingosine kinase genomic DNA transcription initiation and determination sites. “Sphingosine kinase” should also be understood to include reference to any other molecules which exhibit the functional activity of sphingosine kinase. Such molecules include, for example, endogenously expressed molecules which exhibit sphingosine kinase functional activity or molecules which have been introduced into the body and which mimic at least one of the sphingosine kinase functions. These molecules may be recombinant, synthetic or naturally occurring. To the extent that it is not specified, any reference to modulating the activity of sphingosine kinase includes modulating the expression of a nucleic acid molecule encoding sphingosine kinase or the functional activity of the sphingosine kinase expression product and should also be understood to include reference to modulating the expression or functional activity or a sphingosine kinase functional equivalent or derivative.

Reference to “modulating the functional activity” of sphingosine kinase should be understood as a reference to up-regulating, down-regulating or otherwise altering any one or more of the functional activities of sphingosine kinase. This includes, for example, modulating the occurrence of one or more of the sphingosine kinase functional activities, modulating the rate at which a given activity is performed, modulating the level at which an activity is performed, modulating the number of activities which. are capable of being performed or modulating the role or extent to which any activity is performed or the nature of an activity. Changes in the activity of sphingosine kinase can be effected by any one of a number of means including, but not limited to, post translational modification, associated proteins or other molecules or translation. Modulating said activity should also be understood to encompass increasing or decreasing the concentration levels of sphingosine kinase (for example by modulating expression of sphingosine kinase).

Without limiting the present invention to any one theory or mode of action, it has been determined that the oncogenic activity of sphingosine kinase is in particular related to its aberrant overexpression. By “overexpression” is meant the up-regulation of intracellular sphingosine kinase to a functional level which is greater than that expressed under the normal physiological conditions for a given cell type or to the up-regulation of sphingosine kinase levels to any level of functionality but where that up-regulation event is one which is artificially effected rather than being an increase which has occurred in the subject cell due to the effects of naturally occurring physiology. It should be understood, however, that the means by which up-regulation is achieved may be artificial means which seek to mimic a physiological pathway—for example introducing a hormone or other stimulatory molecule Accordingly, the term “expressing” is not intended to be limited to the notion of sphingosine kinase gene transcription and translation. Rather, it is a reference to an outcome, being the establishment of a higher functional level of sphingosine kinase than is found under normal physiological conditions in a cell at a particular point in time (i.e. it includes non-naturally occurring increases in sphingosine kinase level and increases in the level of activity of existing sphingosine kinase concentrations as opposed to just increases in intracellular concentrations, per se, of sphingosine kinase). Accordingly, in a preferred embodiment the subject functional activity is down-regulated from a level of “overexpression” to a level which is not overexpressed (“oncogenic ineffective level”) in order to down-regulate the growth of a neoplastic cell. Conversely, if it is desired to induce a neoplastic state, the level of sphingosine kinase is up-regulated to a level concomitant with “overexpression” (“oncogenic effective level”).

Accordingly, the present invention more preferably provides a method of modulating the growth of a neoplastic cell, said method comprising contacting said cell with an effective amount of an agent for a time and under conditions sufficient to modulate the level of functional activity of sphingosine kinase wherein down-regulation of the functional activity of said sphingosine kinase to an oncogenic ineffective level down-regulates said growth and up-regulation of the functional activity of said sphingosine kinase to an oncogenic effective level up-regulates said cell growth.

As detailed above, the present invention is predicated on the identification of a correlation between sphingosine kinase functional activity and cell growth, in particular, oncogenic cell proliferation. Without limiting the present invention to any one theory or mode of action, the inventors have found that the signalling cascade stimulated by the lipid kinase, sphingosine kinase, has a major role in oncogenesis. Specifically, constitutive activation of sphingosine kinase by overexpression in cells causes cell transformation and tumour formation, thereby indicating that a wild type human lipid kinase is by itself oncogenic.

Furthermore, sphingosine kinase is also involved in Ras but not v-Src induced transformation. Finally inhibition of sphingosine kinase activity utilising a sphingosine kinase inhibitor not only reverses tranformation in cells overexpressimg sphingosine kinase but does so also in Ras transformed cells. In this regard, reference to “modulating” the growth of a cell should be understood as a reference to up-regulating or down-regulating the growth of a cell. More specifically, reference to “down-regulating” should be understood as a reference to preventing, reducing or otherwise inhibiting one or more aspects of the growth of a cell (including inducing the apoptosis of or otherwise killing a cell) while reference to “up-regulating” should be understood to have the converse meaning, and includes induction of the formation of neoplastic cells/cellular transformation (i.e. the conversion of a normal cell to a neoplastic cell). Reference to the “growth” of a cell should be understood in its broadest sense to include reference to all aspects of cell division/proliferation and/or differentiation. In a particularly preferred embodiment, the subject growth is proliferation.

According to this preferred embodiment, there is provided, a method of modulating the proliferation of a neoplastic cell, said method comprising contacting said cell with an effective amount of an agent for a time and under conditions sufficient to modulate the functional activity of sphingosine kinase wherein down-regulation of the functional activity of said sphingosine kinase to an oncogenic ineffective level down-regulates said growth and up-regulation of the functional activity of said sphingosine kinase to an oncogenic effective level up-regulates said cell growth.

Still more preferably, there is provided a method of down-regulating the proliferation of a cell, said method comprising contacting said cell with an effective amount of an agent for a time and under conditions sufficient to down-regulate the functional activity of sphingosine kinase to an oncogenic ineffective level.

In still another preferred embodiment, there is provided a method of up-regulating the proliferation of a cell, said method comprising contacting said cell with an effective amount of an agent for a time and under conditions sufficient to up-regulate the functional activity of sphingosine kinase to an oncogenic effective level.

In a most preferred embodiment said proliferation is uncontrolled proliferation such as that caused by the transformation of a cell. Preferably said transformation is caused by the upregulation of an oncogene such as Ras or by sphingosine kinase overexpression oncogenic activity.

Preferably said functional activity is the level of sphingosine kinase functional activity.

It should be understood that reference to a “cell” in the context of the present invention is a reference to any form or type of cell, irrespective of its origin. For example, the cell may be a naturally occurring cell or it may be manipulated, modified or otherwise treated either in vitro or in vivo such as a cell which has been freezed/thawed or genetically, biochemically or otherwise modified either in vitro or in vivo (including, for example, cells which are the result of the fusion of two distinct cell types). By “neoplastic cell” is meant a cell exhibiting uncontrolled proliferation. The neoplastic cell may be a benign cell or a malignant cell. Preferably the cell is malignant. In one particular embodiment, the neoplastic cell is a malignant cell the proliferation of which would form a solid tumour such as a malignant cell derived from the mammary gland (breast), colon, stomach, lung, brain, bone, oesophagus or pancreas.

According to this most preferred embodiment there is provided a method of down-regulating the proliferation of a neoplastic cell, said method comprising contacting said neoplastic cell with an effective amount of an agent for a time and under conditions sufficient to down-regulate the functional activity of sphingosine kinase to an oncogenic ineffective level.

Preferably the neoplastic cell is a malignant cell derived from the colon, stomach, lung, brain, bone, oesophagus, pancreas, mammary gland (breast), ovary or uterus.

Most preferably, said cell is a mammary cell.

In the context of this preferred embodiment and without limiting the present invention in any way, it has been determined that estrogen activates a signalling pathway via activation of sphingosine kinase in breast cancer cells. Estrogen has dual actions to stimulate sphingosine kinase activity, i.e. a rapid and transient activation mediated by putative membrane G protein-coupled ER and a delayed but prolonged activation relying on the transcriptional activity of ER. The estrogen-induced sphingosine kinase activity consequently activates down-stream signal cascades including intracellular Ca²⁺ mobilization and Erk1/2 activation. Enforced expression of human sphingosine kinase type 1 gene in breast cancer cells results in increases in sphingosine kinase activity and significantly stimulates cell growth. Moreover, the estrogen-dependent mitogenesis and oncogenesis are highly promoted by sphingosine kinase overexpression. In contrast, expression of sphingosine kinase^{G82D}, a dominant-negative mutant sphingosine kinase, profoundly inhibits the estrogen-mediated cell growth and transforming activity in breast cancer cells. However, it should be understood that the present invention should not be limited in this way since it also potentiates estrogen-independent breast cancer cell growth Accordingly, in a preferred embodiment said breast neoplasm is an estrogen-dependent neoplasm.

Still without limiting the present invention to any one theory or mode of action, the mammary gland is a structurally dynamic organ which varies with age, menstrual cycle and reproductive status. It is a branched tubuloalveolar gland exhibiting secretory acinii which are grouped with inner lobules and drain into intralobular ducts which in turn drain into interlobular ducts. The lobules are organised into 15-20 lobes, each of which empty into separate lactiferous sinuses and from there into lactiferous ducts. The intralobular stroma consists of a loose connective tissue with a zone of hormone sensitive fibroblasts surrounding the lobular epithelial components. These are thought to take part in epithelial/basement membrane/stromal inductive interactions during morphogenesis and differentiation. The mammary gland undergoes unique differentiative and proliferative development during the various life cycle stages of an individual. Accordingly, it should be understood that reference to “mammary cells” is a reference to the cells comprising the mammary gland at any stage of its development including prepubescent, pubescent, prenatal, postnatal/lactating and post-menopausal stages. In this regard, it should also be understood that any given population of cells of interest may only be transiently present in the mammary gland, such as those which are generated during pregnancy for the purpose of facilitating lactation.

It should be understood that the cell which is treated according to the method of the present invention may be located ex vivo or in vivo. By “ex vivo” is meant that the cell has been removed from the body of a subject wherein the modulation of its growth will be achieved in vitro. For example, the cell may be a non-neoplastic cell which is to be immortalised by up-regulating sphingosine kinase activity. In accordance with the preferred aspects of the present invention, the cell may be a neoplastic cell, such as a malignant cell, located in vivo (such as in the colon) and the down-regulation of its growth will be achieved by applying the method of the present invention in vivo to down-regulate the level of sphingosine kinase functional activity. It should also be understood that where reference is made to a specific cell type which is located in vivo, such as a malignant colorectal cell, this cell may be located in the colorectal area of the patient. If a colorectal primary malignancy has metastasised, the subject colorectal cell may be located in another region of the patient's body. For example, it may form part of a secondary tumour (metastasis) which is located, for example, in the liver, lymph node or bone.

Although the preferred method is to down-regulate the proliferation of a neoplastic cell, for example as a therapeutic treatment for cancer, it may also be desirable to up-regulate cell growth. For example, it may be desirable to immortalise a population of cells in vitro, to facilitate their long term in vitro use or, for example, to facilitate the in vitro growth of tissues such as skin. In another example, it may be useful to adapt cell lines to less fastidious growth conditions such as a capacity to grow in low serum conditions.

According to this preferred embodiment, the present invention provides a method of up-regulating the proliferation of a cell, said method comprising contacting said cell with an effective amount of an agent for a time and under conditions sufficient to up-regulate the functional activity of sphingosine kinase to an oncogenic effective level.

Preferably, said functional activity is the level of sphingosine kinase functional activity. Without limiting this aspect of the present invention to any one theory or mode of action, said up-regulation of cellular proliferation is preferably transformation of the cell via the up-regulation of sphingosine kinase oncogenic activity.

In this regard, an “effective amount” means an amount necessary to at least partly attain the desired effect, or to delay the onset of, inhibit the progression of, or halt altogether, the onset of progression of a disease condition which is being treated. Such amounts will depend, of course, on the particular conditions being treated, the severity of the condition and individual patient parameters including age, physical conditions, size, weight and concurrent treatment. These factors are well known of those of ordinary skill in the art and can be addressed with no more than routine experimentation. It is preferred generally that a maximum dose be used, that is, the highest safe dose according to sound medical judgement. It will be understood by those of ordinary skill in the art, however, that a lower dose or tolerable dose may be administered for medical reasons, psychological reasons or for virtually any other reasons.

Modulation of the activity of sphingosine kinase by the administration of an agent to a cell can be achieved by one of several techniques including, but in no way limited to, introducing into said cell an agent (said agent being a proteinaceous or non-proteinaceous molecule) which directly or indirectly:
(i) modulates the expression of sphingosine. kinase; or
(ii) modulates the functional activity of sphingosine kinase expression product.

In this regard, modulation of the functional activity of sphingosine kinase can be achieved by any one of several techniques, including, but in no way limited to, introducing into said cell a proteinaceous or non-proteinaceous molecule which directly or indirectly:
(i) modulates synthesis of said sphingosine kinase;
(ii) functions as an antagonist to said sphingosine kinase;
(iii) functions as an agonist to said sphingosine kinase (including administration sphingosine kinase expression product per se or functional equivalent, derivative, homologue, analogue or mimetic thereof).

Said proteinaceous molecule may be derived from natural, recombinant or synthetic sources including fusion proteins or following, for example, natural product screening. Said non-proteinaceous molecule may be derived from natural sources, such as for example natural product screening or may be chemically synthesised. The present invention contemplates chemical analogs of said sphingosine kinase capable of acting as agonists or antagonists of said sphingosine kinase. Chemical agonists may not necessarily be derived from said sphingosine kinase but may share certain conformational similarities. Alternatively, chemical agonists may be specifically designed to mimic certain physiochemical properties of said sphingosine kinase. Antagonists may be any compound capable of blocking, inhibiting or otherwise preventing said sphingosine kinase from carrying out its normal biological functions (for example N,N-dimethylsphingosine or DL-threo-dihydrosphingosine). Antagonists include monoclonal antibodies specific for said sphingosine kinase, or parts of said sphingosine kinase, and antisense nucleic acids which prevent transcription or translation of genes or mRNA in the subject cells. Modulation of expression may also be achieved utilising antigens, RNA, ribosomes, DNAzymes, RNA aptamers, antibodies or molecules suitable for use in co-suppression.

Said proteinaceous or non-proteinaceous molecule may act either directly or indirectly to modulate the expression of sphingosine kinase or the activity of the sphingosine kinase expression product. Said molecule acts directly if it associates with the sphingosine kinase nucleic acid molecule or expression product to modulate expression or activity. Said molecule acts indirectly if it associates with a molecule other than the sphingosine kinase nucleic acid molecule or expression product which other molecule either directly or indirectly modulates the expression or activity of the sphingosine kinase nucleic acid molecule or expression product. Accordingly, the method of the present invention encompasses regulation of sphingosine kinase nucleic acid molecule expression or expression product functional activity via the induction of a cascade of regulatory steps.

Still without limiting the operation of the present invention to any one theory or mode of action, sphingosine kinase is known to function via a signalling pathway which is commonly referred to as the sphingosine kinase signalling pathway. The “sphingosine kinase signalling pathway” is defined as a signalling pathway which utilises sphingosine kinase. In terms of indirect modulation of sphingosine kinase functional activity, it should be understood that the object of the invention could be achieved by modulating the activity of sphingosine kinase signalling pathway components which function either upstream or downstream of sphingosine kinase, to the extent that it forms part of this pathway. For example, modulation of said “sphingosine kinase activity” may be achieved by:
(i) modulation of the catalytic activity of sphingosine kinase by competition with substrate (for example, sphingosine or ATP);
(ii) interference with the catalytic activity of sphingosine kinase by an allosteric mechanism (binding to sites on the molecule other than the substrate-binding sites); or
(iii) interfering with enzyme activation, such as by altering:
   post-translational covalent modification such as phosphorylation, lipid modification
   non-covalent coupling to a required co-activator such as a protein, lipid or ion
   subcellular localisation of the enzyme.

“Derivatives” include fragments, parts, portions, mutants, variants and mimetics from natural, synthetic or recombinant sources including fusion proteins. Parts or fragments include, for example, active regions of sphingosine kinase. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Reference to “homologues” should be understood as a reference to sphingosine kinase nucleic acid molecules or proteins derived from species other than the species being treated.

Chemical and functional equivalents of sphingosine kinase nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Due | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyolodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dummet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to the treatment and/or prophylaxis of disease conditions. Without limiting the present invention to any one theory or mode of action, the inventors have determined not only that constitutive activation of sphingosine kinase causes cell transformation and tumour development, thereby indicating that sphingosine kinase is by itself oncogenic, but that sphingosine kinase inhibition is also effective in down-regulating neoplastic cell proliferation where the subject cell has been transformed by certain unrelated oncogenes such as Ras induced transformation. Accordingly, the method of the present invention is particularly useful, but in no way limited to, use in the treatment of primary and secondary malignancies such as those associated with solid tumours of the colon, stomach, lung, mammary gland (breast), brain, bone, oesophagus and pancreas and, in particular, tumours which arise from the proliferation of Ras transformed cells or estrogen-dependent breast cell tumours. Although the preferred method is to down-regulate uncontrolled cellular proliferation in a subject, up-regulation of cell growth may also be desirable in certain circumstances such as to promote wound healing, angiogenesis or other healing process.

Accordingly, the present invention contemplates a method for the treatment and/or prophylaxis of a condition characterised by aberrant, unwanted or otherwise inappropriate cell growth in a mammal, said method comprising administering to said mammal an effective amount of an agent for a time and under conditions sufficient to modulate the functional activity of sphingosine kinase wherein down-regulation of the functional activity of said sphingosine kinase down-regulates the subject cell growth and up-regulation of the functional activity of said sphingosine kinase up-regulates the subject cell growth.

Reference to “aberrant, unwanted or otherwise inappropriate” cell growth should be understood as a reference to over active cell growth, to physiologically normal cell growth which is inappropriate in that it is unwanted or to insufficient cell growth. Preferably, said inappropriate cell growth is uncontrolled cell proliferation induced by sphingosine kinase overexpression.

According to this preferred embodiment, there is provided a method for the treatment and/or prophylaxis of a condition characterised by uncontrolled cell proliferation in a mammal, said method comprising administering to said mammal an effective amount of an agent for a time and under conditions sufficient to down regulate the functional activity of sphingosine kinase to an oncogenic ineffective level.

Preferably said uncontrolled cell proliferation is caused by the transformation of the cell by oncogene up-regulation or by sphingosine kinase overexpression oncogenic activity.

Still more preferably said cell is a malignant cell which forms a solid tumour of the colon, stomach, lung, brain, bone, oesophagus, pancreas, mammary gland (as or uterus.

Most preferably said cell is a malignant breast cell and still more preferably said malignancy is estrogen-dependent.

Preferably, said agent is N,N-dimethylspingosine or DL-threo-dihydrophingosine.

The method of the present invention preferably facilitates the subject proliferation being reduced, retarded or otherwise inhibited. Reference to “reduced, retarded or otherwise inhibited” should be understood as a reference to inducing or facilitating the partial or complete inhibition of cell proliferation. Said inhibition may occur by either direct or indirect mechanisms and includes the induction of cellular apoptosis or other cellular killing mechanisms.

The subject of the treatment or prophylaxis is generally a mammal such as but not limited to human, primate, liverstock animal (eg. sheep, cow, horse, donkey, pig), companion animal (eg. dog, cat), laboratory test animal (eg. mouse, rabbit, rat, guinea pig, hamster), captive wild animal (eg. fox, deer). Preferably the mammal is a human or primate. Most preferably the mammal is a human. Although the present invention is exemplified utilising a murine model, this is not intended as a limitation on the application of the method of the present invention to other species, in particular, humans.

Another aspect of the present invention relates to the treatment and/or prophylaxis of a neoplastic condition in a mammal, said method comprising administering to said mammal an effective amount of an agent for a time and under conditions sufficient to down-regulate the functional activity of sphingosine kinase to a functionally ineffective level.

Preferably said neoplastic condition is a malignant condition and even more preferably a solid malignancy such as a tumour of the colon, stomach, breast, lung, brain, bone, oesophagus or pancreas.

More preferably said neoplastic condition is breast cancer and still more preferably an estrogen-dependent neoplastic condition.

Still more preferably the subject malignancy is caused by transformation of the cell via oncogene up-regulation or by sphingosine kinase overexpression oncogenic activity.

Still more preferably said agent is N,N-dimethylsphingosine or DL-threo-dihydrophingosine.

Reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis including amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Administration of the agent (including sphingosine kinase or functional equivalent, derivative, homologue, analogue or mimetic thereof or sphingosine kinase nucleic acid molecule) [herein referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation. The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In another aspect the present invention relates to the use of an agent capable of modulating the functional activity of sphingosine kinase in the manufacture of a medicament for the modulation of neoplastic cell growth in a mammal wherein down-regulation of the functional activity of said sphingosine kinase down-regulates the subject cell growth and up-regulation of the functional activity of said sphingosine kinase up-regulates the subject cell growth.

Preferably said cell growth is proliferation.

Preferably said uncontrolled cell proliferation is caused by the transformation of the cell by oncogene up-regulation or by sphingosine kinase overexpression oncogenic activity.

Still more preferably said cell is a malignant cell which forms a solid tumour of the colon, stomach, lung, brain, bone, oesophagus, pancreas, mammary gland (breast), ovary or uterus.

Most preferably said cell is a breast cell.

Still more preferably, said breast cell malignancy is estrogen-dependent.

Yet another aspect relates to agents for use in modulating the functional activity of sphingosine kinase wherein modulating the functional activity of sphingosine kinase modulates neoplastic cell growth.

Preferably said functional activity is down-regulated thereby down-regulating neoplastic cell growth.

In another preferred embodiment said sphingosine kinase functional activity is up-regulated thereby up-regulating neoplastic cell growth.

Even more preferably said cell growth is cell proliferation.

Preferably said cell proliferation is caused by the transformation of the cell by oncogene up-regulation or by sphingosine kinase overexpression oncogenic activity.

Still more preferably said cell is a malignant cell which forms a solid tumour of the colon, stomach, lung, brain, bone, oesophagus, pancreas, mammary gland (breast), ovary or uterus.

In yet another aspect the present invention relates to a pharmaceutical composition comprising a modulatory agent as hereinbefore defined and one or more pharmaceutically acceptable carriers and/or diluents. Said modulatory agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible cattier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Screening for the modulatory agents hereinbefore defined can be achieved by any one of several suitable methods known to those of skill in the art including, but in no way limited to, contacting a cell comprising the sphingosine kinase gene or functional equivalent or derivative thereof with an agent and screening for the modulation of sphingosine kinase protein production or functional activity, modulation of the expression of a nucleic acid molecule encoding sphingosine kinase or modulation of the activity or expression of a downstream sphingosine kinase cellular target. Detecting such modulation can be achieved utilising techniques such as Western blotting, electrophoretic mobility shift assays and/or the readout of reporters of sphingosine kinase activity.

It should be understood that the sphingosine kinase gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate sphingosine kinase activity, at either the nucleic acid or expression product levels, or the gene may require activation—thereby providing a model useful for, inter alia, screening for agents which up regulate sphingosine kinase expression. Further, to the extent that a sphingosine kinase nucleic acid molecule is transfected into a cell, that molecule may comprise the entire sphingosine kinase gene or it may merely comprise a portion of the gene such as the portion which regulates expression of the sphingosine kinase product. For example, the sphingosine kinase promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream sphingosine kinase regulatory target, rather than sphingosine kinase itself. Such detection may be achieved utilising technologies such as microarrays (e.g. Chip arrays, nylon arrays), SAGE analysis, RDA or Differential Display.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the sphingosine kinase nucleic acid molecule or expression product itself or which modulate the expression of an upstream molecule, which upstream molecule subsequently modulates sphingosine kinase expression or expression product activity. Accordingly, these methods provide a mechanism of detecting agents which either directly or indirectly modulate sphingosine kinase expression and/or activity.

As detailed earlier, reference to “sphingosine kinase” should be understood as a reference to either the sphingosine kinase expression product or to a nucleic acid molecule encoding sphingosine kinase. It should also be understood as a reference to a portion or fragment of the sphingosine kinase molecule such as the regulatory region of the sphingosine kinase nucleic acid molecule. Alternatively, the molecule may comprise the binding/active portion of the expression product. In this regard, the sphingosine kinase nucleic acid molecule and/or expression product is expressed in a cell. The cell may be a host cell which has been transfected with the sphingosine kinase nucleic acid molecule or it may be a cell which naturally contains the sphingosine kinase gene.

The screening method herein defined may be based on detecting an “altered expression phenotype associated with said sphingosine kinase”. This should be understood as the detection of cellular or cell culture condition changes associated with modulation of the activity of sphingosine kinase. These may be detectable for example as intracellular changes or changes observable extracellularly. For example, this includes, but is not limited to, detecting changes in expression product levels, cell culture condition changes or, to the extent that the sphingosine kinase regulatory region is ligated to a reporter molecule such as luciferase or CAT, detecting changes in reporter molecule expression. Alternatively, this screening system may be established to detect changes in the expression of downstream molecules which are regulated by the sphingosine kinase expression product.

Yet another aspect of the present invention relates to modulatory agents, as hereinbefore defined, when used in the method of the present invention.

Still yet another aspect of the present invention relates to diagnostic methodology based on screening individuals for the presence of sphingosine kinase or mRNA or protein or the specific forms of sphingosine kinase which are transcribed and/or translated by a given population of cells. The screening methodology may be directed to qualitative and/or quantitative sphingosine kinase analysis. This is particularly useful, for example, for determining whether a given individual is predisposed to or resistant to diseases/disorders in which aberrant, unwanted or otherwise inappropriate cell growth is a component of the disease state and/or is predisposed or resistant to the development of certain forms of aberrant, unwanted or otherwise inappropriate cell growth. Such screening can be performed utilising methods which would be known to those skilled in the art including, but not limited to:
(i) The use of sphingosine kinase assays to screen for altered sphingosine kinase activity or levels. These parameters can be screened for either in the bodily fluids or tissues of individuals. Although sphingosine kinase is generally not secreted, its down-stream product sphingosine kinase-1-phosphate is secreted. Modulation of the levels of this molecule could therefore be used as an indicator of changes in sphingosine kinase levels or activity. Nevertheless, screening for extracellular sphingosine kinase should not be excluded.
(ii) Analysis of sphingosine kinase for possible mutations/polymorphisms (SNPs) and mutations can be performed by melting curve analysis of PCR generated DNA using the LightCycler system (Roche).
(iii) The screening of SNPs involving sphingosine kinase using Chip Arrays (e.g. Affimetrix GeneChipSNP mapping or using the Incyte technology platform fSSCP for SNP discovery.

The present invention is further described by the following non-limiting examples.

### EXAMPLE 1

### Transfection and Analysis of Oncogene Transformed Cells

Materials and Methods

(a) Sphingosine Kinase Transfection

NIH 3T3 fibroblasts were obtained from the American Type Culture Collection and maintained in DMEM (GIBCO BRL), supplemented with 10% calf serum. The human SphK cDNA tagged with a FLAG tag was subcloned into the pcDNA3 plasmid (Invitrogen Corp.) as previous described (Pitson, S. M., Moretti, P. A., Zebol, J. R., Xia, P., Gamble, J. R., Vadas, M. A., D'andrea, R. J., and Wattenberg, B. W. (2000) J Biol Chem, 275: 33945-33950.). For transient transfections, 5 μg of plasmids were transfected to 5×10⁵ using Lipofectamine Plus (GIBCO BRL) according to the manufacturer's protocols. For stable expression, the calcium phosphate precipitation method was used and the transfectants were selected in medium containing 500 μg/ml G418 (GIBCO BRL). The nonclonal pools of G418-resistant transfected cells were collected and used to avoid clonal variability. For some experiments, the selected clones of stable transfectants were used.

(b) Measurement of Sphingosine Kinase Activity

SphK activity was measured by incubating the cytosolic fraction with 10 μM sphingosine dissolved in 5% Triton X-100 and [(³²P]ATP (1 mM, 0.5 mCi/ml) for 15 min at 37° C. as described previously (Xia, P., Vadas, M. A., Rye, K. A., Barter, P. J., Gamble, J. R. (1999) J. Biol. Chem. 274:33143-33147). For assay of intracellular level of S1P, cells were labelled with [³H]sphingosine (1 μM, 2 μCi/ml) for 30 min and radioactivity incorporated into cellular lipids were extracted. [³H]S1P was then resolved on TLC with 1-butanol/methanol/acetic acid/water (8:2:1:2, vol/vol), visualized and quantified by Phosphoimager®.

(c) Culture of Sphingosine Kinase-3T3 Cells

Stably transfected NIH 3T3 cells were plated in 48-well plates (1,000 cells per well) in DMEM containing 10% calf serum. After 8 h, cells were washed twice with DMEM and then grown in DMEM containing 1 or 10% serum and serum-free medium (DMEM containing 0.1% BSA). At the indicated times, cells were incubated with 1 mg/ml MTT for 4 h. The formazan product was solubilized by 10% in SDS in 10 mM HCl and assessed by spectrophotometry at 570 nm and 650 nm absorbance. In some experiments, cells were trypsinzed and counted in a hemocytometer.

(d) Focus Formation Assay

For focus formation assay, low passage NIH 3T3 cells were transfected with the activated V12 Ras, v-Src (gifts of Dr. Julian Downward), SphK, or empty vector, respectively, using Lipofectamine Plus as described in (a) above. Two days later, the transfected cells were split to 6-well plates. After reaching confluence, they were kept for two weeks in DMEM containing 5% calf serum. The foci were visualized and scored after stained with 0.5% crystal violet. For soft agar assay, suspensions of 1×10⁴ cells from the stable transfected pools in a growth medium containing 0.33% agar were overlaid onto 0.6% agar gel in the absence or presence of DMS at various concentrations. After a 14-day incubation colonies were stained with 0.1 mg/ml MTT and that over 0.1 mm in diameter were scored as positive.

(e) Tumour Induction

NOD/SCID mice were bred and maintained under sterile conditions. Four- to six-week-old mice were injected subcutaneously with 5×10⁵ cells in 200 μl sterile PBS from various cell lines (stable SphK- and vector-transfected NIH 3T3 cell pools, two colonies of SphK transfectants). Each cell line was tested in 3 different animals.

Results

(a) Transfection and Analysis of NIH 3T3 Fibroblasts

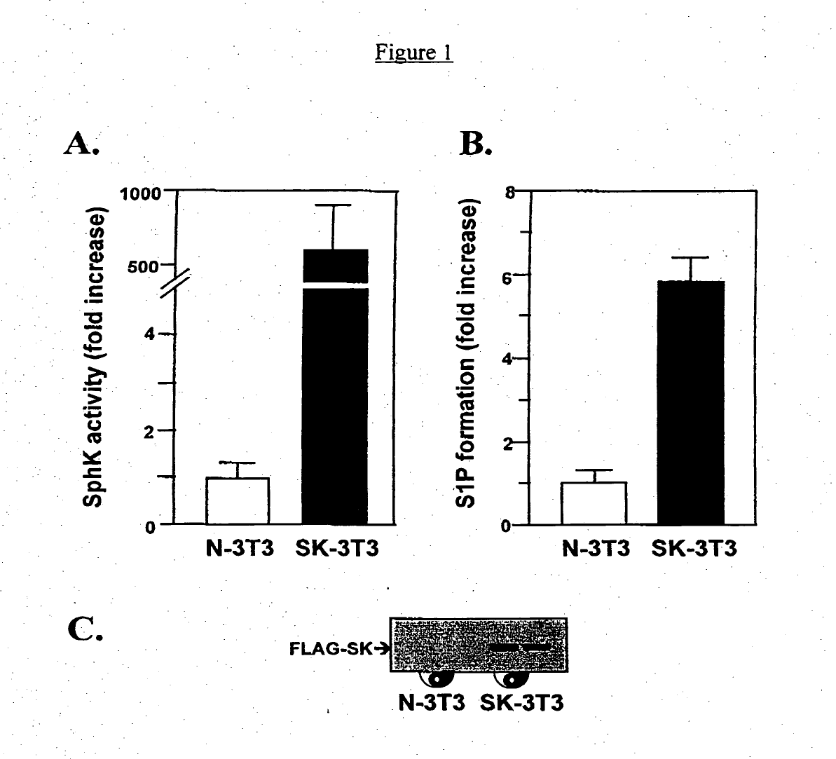

To investigate the oncogenic role of SphK, the nontransformed NIH 3T3 fibroblasts were transfected with human SphK cDNA that was recently cloned in our laboratory (Pitson et al., 2000 supra). Pooled stable transfectants (referred as SK-3T3) were used to avoid the phenotypic artifacts that may due to the selection and propagation of individual clones from single transfected cells. In the SK-3T3 cell pools SphK activity was increased by over 600-fold (FIG. 1a) in comparison with the empty vector-transfected NIH 3T3 cells (N-3T3). Immunoblotting analysis showed a specific protein band with an apparent molecular weight consistent with the predicted size of FLAG-tagged human SphK that was detected only in the SK-3T3 cell pools but absent in N-3T3 cells (FIG. 1c). Intracellular levels of S1P, the direct product of SphK, were also increased in SK-3T3 cells by 4- to 5-fold, indicating the stable transfectants with constitutively activation of SphK (FIG. 1b). S1P levels were not directly proportional to the increase in SphK activity assayed in vitro perhaps due to rapid degradation of S1P by S1P phosphatase and S1P lysase and the limited availability of sphingosine as a substrate for S1P formation.

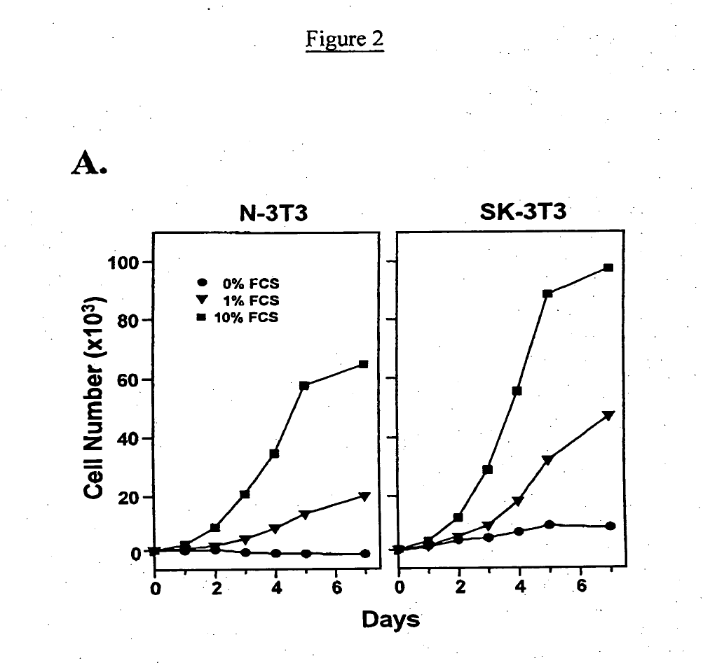

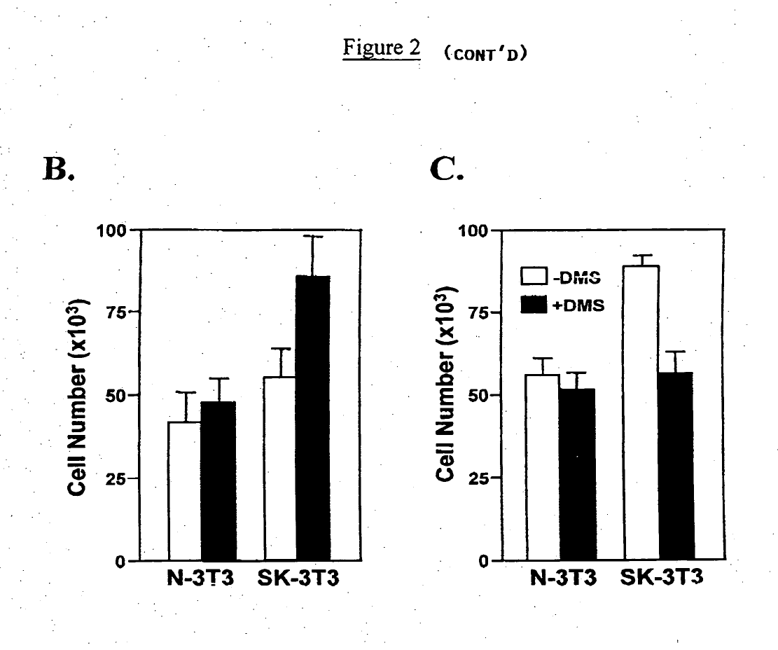

Growth curves showed a significant difference between the SK-3T3 cell pools and the controls (FIG. 2a). Stable expression of SphK dramatically enhanced cell growth in the media containing either 1% or 10% serum. Even in serum-free medium for up to 7 days, SK-3T3 cells survived and grew whilst the N-3T3 cells underwent death. Furthermore, when the cells reached saturation density SK-3T3 cells continued to proliferate (FIG. 2b) suggesting an escape from contact inhibition. Treatment of SK-3T3 cells with a specific inhibitor of SphK, N,N-dimethylsphingosine (DMS), significantly diminished the enhanced proliferation induced by overexpression of SphK, whilst DMS had no effect on proliferation of N-3T3 cells (FIG. 2c). These results indicate that the constitutive activation of SphK in the stably SphK-transfected cells reduces two key growth liming properties: serum dependence and contact inhibition.

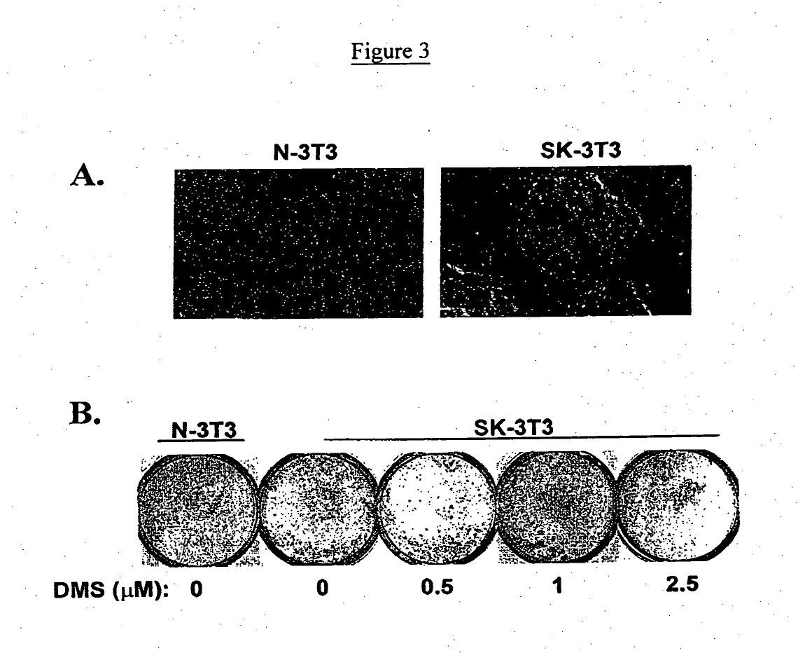

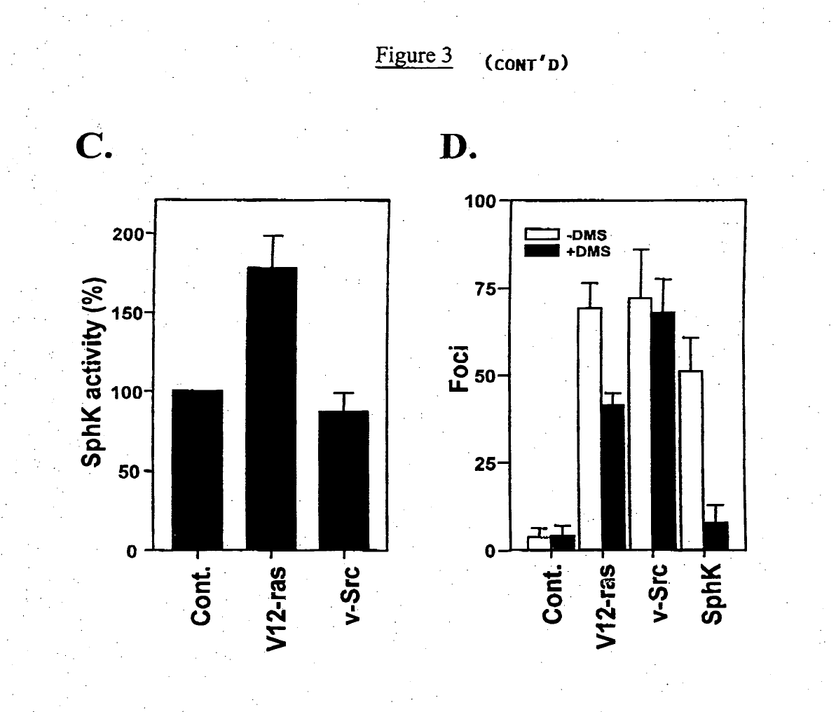

The transforming activity assayed by focus formation in NIH 3T3 cells showed that cells transfected with SphK but not empty vector induced numerous foci (Table 1 and FIG. 3a). Both SphK and control vectors displayed similar efficiency in the generation of G418-resistant colonies indicating that the transforming activity was not due to non-specific effect of transfection. Furthermore,. SK-3T3 cells formed vigorous colonies in soft agar (Table 2 and FIG. 3b) revealing the acquisition of anchorage-independent growth. Although N-3T3 cells exhibited a background level of colony formation that may due to spontaneous transformation, overexpression of SphK resulted in a 20˜50-fold increase in the number of colonies and an obvious increase in colony size (Table 2), Importantly, DMS inhibited the transforming capacity of SphK in a dose-dependent manner with 2.5 μM DMS resulting in total reversion to the normal phenotype (FIG. 3b). This suggested that the increased activity of SphK rather than its mere overexpression is responsible for the transforming capacity of this enzyme.

(b) Transfection and Analysis of Oncogene Transformed Cells

When NIH 3T3 cells were transfected with an activated mutant Ras (V1 2-Ras), SphK activity was significantly increased by 178±22% in comparison to the parent cells (FIG. 3c). By contrast the cells transfected with v-Src (FIG. 3c) or dominant-negative Ras (N17-Ras had no changes in the activity of SphK, suggesting a specific involvement of SphK in some oncogenic transformation. Moreover, when the cells were treated with DMS, the focus formation was reduced by 42±4% in V12-Ras transformed cells but there were no changes in v-Src transformed cells (FIG. 3d), indicating an important role of SphK in Ras transformation. The likelihood that there are SphK independent pathways operating is suggested by the partial effect of DMS at the dose (2.5 μM) that was fully effective in SphK transformed cells (FIG. 3d). On the other hand, the inability of DMS to inhibit v-Src transformation rules out non-specific effects of DMS or a general toxicity resulting from inhibition of SphK. The increased SphK activity thereby exerts a transforming potential not only in its own right by overexpression but is also involved in oncogenic transformation, eg., induced by Ras.

(c) Analysis of Tumorigenicity

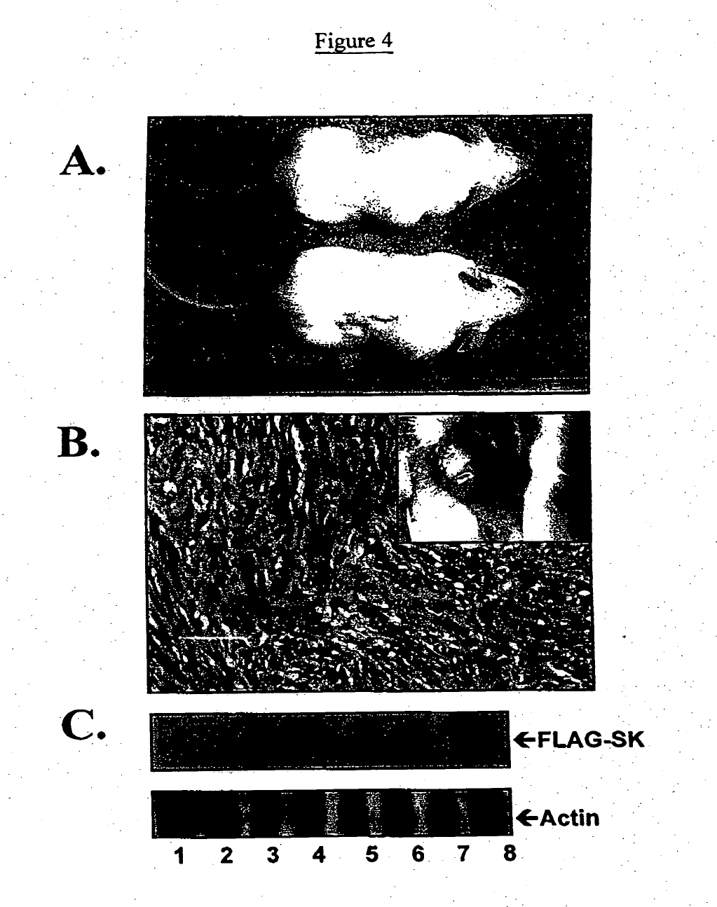

Tumorigenicity was then directly tested in NIH 3T3 cells overexpressing SphK. When the SphK-transfected NIH 3T3 cells from either the stable transfectant pools or selected clones were injected subcutaneously into NOD/SCID mice, tumours became apparent at the site of injection within 3 to 4 weeks (Table 4 and FIG. 4). No mice injected with the vector-transfected 3T3 cells induced tumours during 10-weeks of observation. Histological appearances of tumour sections displayed the morphologies of fibrosarcoma with many mitotic figures (FIG. 4). Western blot analysis of extracts derived from tumours showed high levels of FLAG-tagged protein (FIG. 4), revealing that the neoplastic cells retain and express the SphK transgenes. Thus, the tumours were developed from the injected SphK-transfected cells but not from spontaneously transformed NIH 3T3. This is the first demonstration that a wild type lipid kinase gene, human SphK, acts as an oncogene providing a potential linkage between this enzyme and mammalian tumour pathogenesis. Thus, our finding extends the understanding of phospholipids, particularly sphingolipids, as signal transducers regulating cellular growth, transformation and oncogenesis.

### EXAMPLE 2

### Sphingosine Kinase Transduces Estrogen Signalling in Human Breast Cancer Cells

Materials and Methods

Materials

D-erythro-Sphingosine, S1P and DMS were purchased from Biomol Research Laboratories Inc. (Plymouth Meeting, Pa.). Pertussis toxin, ATP, 17β-estrodiol, 17β-estrodiol-BSA and 4-hydroxytamoxifen (OH-Tam) were from Sigma Chemical Co. ICI 192,780 was purchased from Jomar Diagnostics (Adelaide, South Australia). PD098059 was obtained from Calbiochem (San Diego, Calif., USA). [γ-³²P]ATP was purchased from Geneworks (Adelaide, South Australia). The antibodies against phosphorylated forms of Erk1/2 as well as total Erk1/2 were purchased from Promega (Madison, Wis.).

Cell Cultures and Transfection.

Strains of the human adenocarcinoma MCF-7 cell line (ERα+/β+) (ATCC # HTB-22) and MDA-MB453 (ERα-/β+) (ATCC# HTB-131) were obtained from the American Type Culture Collection. Cells were cultured on phenol red-free Dulbecco's modified Eagle's medium (CSL Biosciences, Parkville, Australia) containing 4.5 g/L glucose, 2mM L-glutamine, 10 mM nonessential amino acids, 1.6 mg/ml penicillin-streptomycin, 10 ng/ml insulin, and 1-10% Fetal Bovine Serum (FBS) (where mentioned). Human SKI (GenBank TM accession number AF200328) and mutant SK^{G82D} cDNA was FLAG epitope-tagged and subcloned into pcDNA3 vector (Invitrogen) as described previously (Pitson, S. M., 2000, supra). Transient transfections were performed using the LipofectAMINE 2000 (Invitrogen) according to the manufacturer's protocols at a cell density of 10⁶ cells/ml with 20 μg of DNA. Stable transfectants were selected in medium containing 0.8 g/L G418 (GIBCO BRL).

Cell Growth Assay.

To assess cell proliferation, we used the CellTiter 96 AQueous proliferation assay (Promega Corp.) that is based on metabolic conversion of a tetrazolium compound MTS to a colored product by living cells as described in the manufacturer's protocols. Cells (4×10³ cell/well) were seeded in 4 to 6 replicates into 96-well plates and incubated at 37° C. in 5% CO₂ with or without E₂ (10 nM) in phenol-red free media for a desired time period. The medium was changed every 2-3 days. The absorbance intensity of the MTS product is directly proportional to the number of viable cells in culture when cell number is between 2000 and 200 000, otherwise the exponential dependence was determined. Total cell numbers were calculated based on calibration curves.

Focus Formation and Colony Formation Assays.

MCF-7 cells were seeded into 24-well plastic tissue culture plates at a density of 1×10⁵ cells/ml/well, and maintained in phenol-red free media containing 10% FCS until the cells reached confluent. The cells were then refed at 24 h before first treatment with or without 10 nM E₂ and then every 2-3 days thereafter with 1% FCS medium. After 5, 10 and 14 days the cultures were photographed and number of foci were counted. For colony assay, cells (1×10⁴) suspended in 2 ml of 0.36% agar (Becton Dickinson, Sparks, MD) with phenol-red free growth medium were added on a base layer of 0.72% agar containing culture medium as described previously (Xia, P., Gamble, J. R., Wang, L., Pitson, S. M., Moretti, P. A., Wattenberg, B. W., D'andrea, R. J., and Vadas, M. A. (2000) Curr. Biol., 10: 1527-1530). After 14 days of incubation, the colonies were stained with MTT and counted. All experiments were done at least three times using triplicates per experimental point. Average was assessed by counting the number of colonies under low magnification (×20) at four points on each well.

Western Blotting.

Cells were harvested and lysed by sonication in lysis buffer containing 50 mM Tris/HCl (pH 7.4), 10% glycerol, 0.5% Triton-X-100, 150 mM NaCl, 1 mM dithiothreitol, 2 mM Na₃VO₄, 10 mM NaF, 1 mM EDTA and protease inhibitors (Complete; Roche Molecular Biochemicals). Protein concentrations of the cell lysates were determined with a Bio-Rad Dc protein assay kit (Bio-Rad Laboratories, CA). Aliquots of cell lysates containing 30-50 μg of proteins were resolved by 12% SDS-PAGE and transferred to Hybond-P membranes (Amersham Biosciences, Inc.). The membranes were then probed with appropriate antibodies according to standard method as described previously (Pitson, S. M., 2000, supra). The immunocomplexes were detected with an enhanced chemiluminescence PLUS kit (Amersham Pharmacia Biosciences) and by using Typhoon 9410 Variable Mode Imager (Amersham Pharmacia Biotechnologies).

SK Activity Assay.

Briefly, after desired treatments the cells were washed with ice-cold PBS, scraped from the culture dishes and homogenized in lysis buffer containing 20 mM Tris (pH 7.4), 20% glycerol, 1 mM DTT, 1 mM EDTA, 10 μM MgCl₂, 1 mM Na₃VO₄, 15 mM NaF, 10 μg/ml leupeptin and aprotinin, 1 mM PMSF and 0.5 mM 4-deoxypyridoxine. After centrifugation at 13,000×g for 30 min, SK activity was measured by incubating the cytosolic fraction with 5 μM sphingosine dissolved in 0.1% Triton X-100 and [γ³²P]ATP (1 mM, 0.5 mCi/ml) for 30 min at 37° C. as described previously (Pitson et al, 2000, supra; Xia, P., 1998, supra). The enzyme activity was normalized by total protein concentration of each sample.

Measurement of [Ca²⁺]ᵢ.

MCF-7 cells (1×10⁸) were trypsinized, washed and resuspended in 1 ml of phenol-red free media without serum and incubated with 2 μM Sulfinpyrazone for 15 min at room temperature. Then cells were incubated for 60 min at 37° C. with 20 μM Fluo-3/AM combined with 20% Pluronic F-127 (1:1) in the dark, washed and removed for immediate analysis on Perkin Elmer luminescence spectrophotometer (LS 50B) (Perkin Elmer Corporation, Norwalk, Conn.). The fluorescence of the cells was monitored at an excitation wavelength of 490 nm and an emission wavelength of 525 nm. Each recording was calibrated by determining the maximal uptake of calcium (Fmax) in the presence of 0.1% Triton X as well as the level of autofluorescence (Fmin), after quenching Fluo-3/AM fluorescence with 1 mM Mn2⁺. Intracellular calcium concentration, [Ca²⁺]ᵢ, was calculated as previously described (Grynkiewicz, G., Poenie, M., and Tsien, R. Y. (1985) J Biol Chem, 260: 3440-3450; Kao, J. P., Harootunian, A. T., and Tsien, R. Y. (1989) J Biol Chem, 264: 8179-8184) using the equation: [Ca²⁺]ᵢ=K_{d} (F-Fmin)/(Fmax-F), where K_{d} is the dissociation constant of fluo-3/AM set to 400 nM.

Results

Expression of Human SK Transgenes in MCF-7 Breast Cancer Cells.

To investigate the effect of SK on human breast cancer cells, MCF-7 cells were stably transfected with constitutively expressed FLAG tagged-human SK1 or a point mutation of SK1 (SK^{G82D}) that lacks the enzymatic activity (Pitson et al, 2000, supra). Pooled stable transfectants were used (unless indicated) to avoid the phenotypic artifacts that may be due to the selection and propagation of individual clones from single transfected cells. Immunoblotting analysis showed specific protein bands detected in both wild type SK and SK^{G82D} transfected cell pools, but absent in the empty vector transfected cells (FIG. 1a). Detected bands demonstrated apparent molecular weight consistent with the predicted size of FLAG-tagged human SK1. Overexpression of SK in the cells resulted in an over 10-fold increase in the basal SK activity, whereas the SK^{G82D} transfected cells had a similar basal SK activity to the control cells transfected with empty vectors alone (FIG. 1b). To confirm the transgenes of wild type SK and SK^{G82D} functionally expressed in MCF-7 cells, SK activity was measured after the cells were stimulated with phorbol 12-myristate 13-acetate (PMA), a known activator of SK through PKC activation (Pitson et al., 2000, supra; Culliver, O., 1996, supra). PMA stimulation resulted in an approximately 2-fold increase in SK activity over the basal levels in both SK and the empty-vector transfected MCF-7 cells (FIG. 1b). No SK activation was observed in the cells expressing SK^{G82D} in response to PMA stimulation (FIG. 1b), confirming that SK^{G82D} acts as a dominant-negative SK in the transfected MCF-7 cells.

SK Overexpression Stimulates E₂-Dependent and -Independent MCF-7 Cell Growth.

Similar to previous results with SK-transfected NIH 3T3 cells (Xia et al., 2000, supra), overexpression of SK in MCF-7 cells dramatically enhanced cell growth FIGS. 2a and 2b). Even in serum-free medium for up to 5 days, SK transfected MCF-7 cells survived and continued to grow (FIG. 2a), indicating a mitogenic effect resulted from SK overexpression.

The effect of SK on E₂-dependent cell growth was also examined. Growth curves shows that treatment MCF-7 cells with E₂ stimulated cell growth in the absence of serum (FIG. 2b). Remarkably, the E₂ responsive cell growth was significantly enhanced in the wild type SK-transfected cells (FIG. 2b). In contrast, the SK^{G82D}-transfected cells not only lost the mitogenic activity of SK, but also dramatically inhibited the effect of E₂ on cell growth (FIG. 2b), indicating a critical role of SK in mediating E₂-dependent mitogenesis in MCF-7 breast cancer cells.

Effect of SK on E₂-Promoted Transforming Activity in MCF-7 Cells.

As shown in FIG. 3a, E₂ stimulation was able to form solid cell foci within 10-day culture of MCF-7 cells. Overexpression of SK resulted in more than 2-fold increases m the number of foci formation compared to the empty vector-transfected cells (FIG. 3a). The size of focus was also markedly increased in the SK-transfected MCF-7 cells. Conversely, under same culture conditions with E₂ treatment, the cells transfected with SK^{G82D} was unable to form foci (FIG. 2a) even prolonged the cultures to 14 days. Additionally, a similar result was obtained in the experiments with the transforming activity assay of colony formation in soft agar (FIG. 3b). These data suggest a requirement of SK activity for the E₂-dependent transforming activity in MCF-7 cells.

E₂ Stimulates SK Activity in MCF-7 Cells.

Given a potential role of SK in E₂-mediated mitogenesis and transformation, it was sought to determine the effect of E₂ on SK activation. As shown in FIG. 4a, E₂ stimulation of MCF-7 cells caused a rapid and transient increase in SK activity, reaching a maximum of 240±25% (p<0.01) of basal within 15 min and returning to background level after 30 min treatment. The E₂-induced SK activity was dose-dependent, reaching a maximum at 1 μM of E₂ (FIG. 4b). To determine prolonged effect of E₂ on SK activity the MCF-7 cells were treated for 1, 6, 18, 24, and 48 hours with 10 nM of E₂. A significant increase in SK activity was detected after 6 hours of E₂-treatment and remained the activity more than 24 hours (FIG. 4c). Thus, two peaks of SK activities were observed in the MCF-7 cells treated with E₂. The first peak happened very quickly within 5-15 min of E₂ stimulation suggesting an nongenomic effect of E₂. The second prolonged peak required at last 6 hours of E₂ exposure and could possibly connected to the genomic effect of ER. In support this hypothesis, pretreatment with actinomycin D, a transcriptional inhibitor, had no overt effect on the first peak of E₂-induced SK activity (FIG. 4d). Whereas, the delayed activation of SK was abrogated by actinomycin D (FIG. 4c), suggesting an involvement of transcriptional activity in the delayed but prolonged effect of E₂ on SK. Furthermore, after pretreatment with E₂ for 16 h cells were pulsed in an additional dose of E₂ (10 nM) resulted in an additive increase in SK activity with a same profile as the primary quick response to E₂ (FIG. 4d). Collectively, these data indicated that E₂ has a dual effect on SK activity dependent on E₂ genotropic and nongenotropic action, respectively.

E₂-Induced SK Activation is Mediated by Putative Membrane ER.

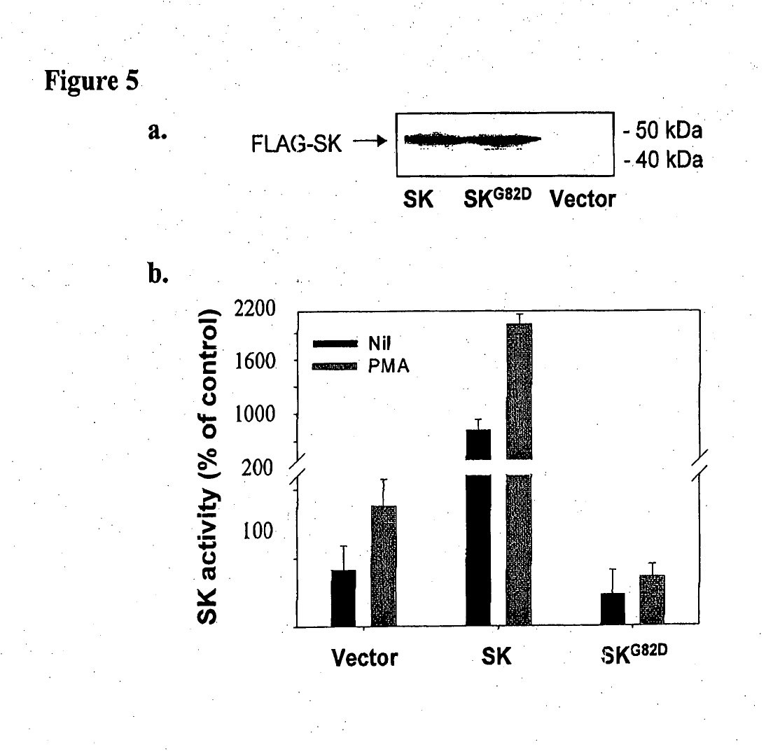

The rapid response of SK activity to E₂ stimulation along with the inability of actinomycin D on this action has suggested a transcription-independent effect of E₂. To pursue this idea further and define which receptor(s) of E₂ might be involved in the E₂-induced SK activity, the effect of E₂ on MDA-MB-231 breast cancer cell line that expressed ERβ but lacks Erα was examined. No increased activity of SK was detected in MDA-MB-231 cells treated with E₂ for either 15 min or 6 hours (FIG. 5a), suggesting that the effect of E₂ on SK activation is independent of ERβ. Then, we used the inhibitors of intracellular ER, Tamoxifen or ICI 182,780, which have been shown to effectively block the transcriptional action of nuclear ER (Hyder, S. M., Chiappetta, C., Murthy, L., and Stancel, G. M. (1997) Cancer Res., 57: 2547-2549). Treatment of ICI 182,780 (FIG. 5b) or Tamoxifen had no influence on E₂-mediated SK activation in MCF-7 cells, further supporting a transcription-independent effect of E₂ on the activation of SK. To verify E₂ signalling through putative membrane ER membrane impermeable E₂-BSA was used. FIG. 5b shows that E₂-BSA had a similar effect to its natural analog E₂ on SK activity in MCF-7 cells. Moreover, pretreatment with pertussis toxin (PTX), a G-protein inhibitor, strongly abrogated E₂-mediated activation of SK (FIG. 5b), implying Gi protein-coupled receptor complexes are responsible for the E₂-induced quick activation of SK.

Effect of SK on E₂-Induced Intracellular Ca²⁺ Mobilization.

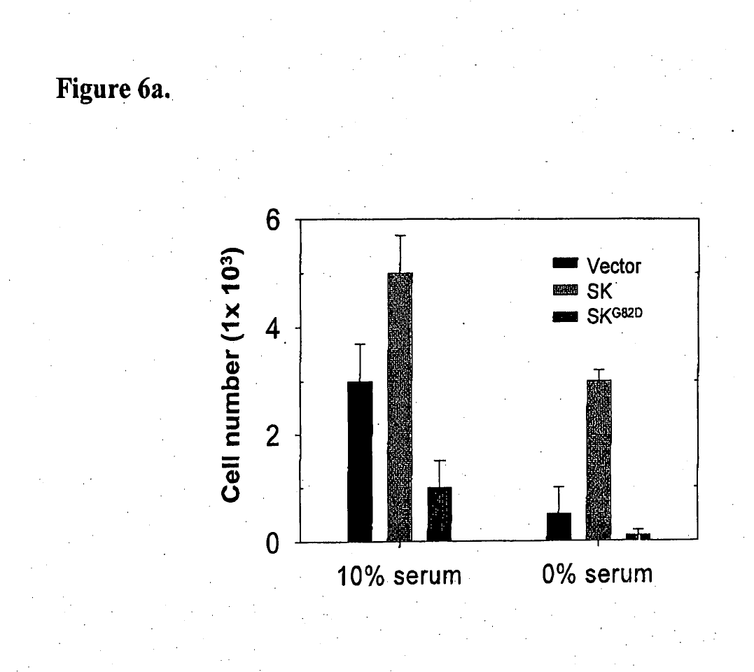

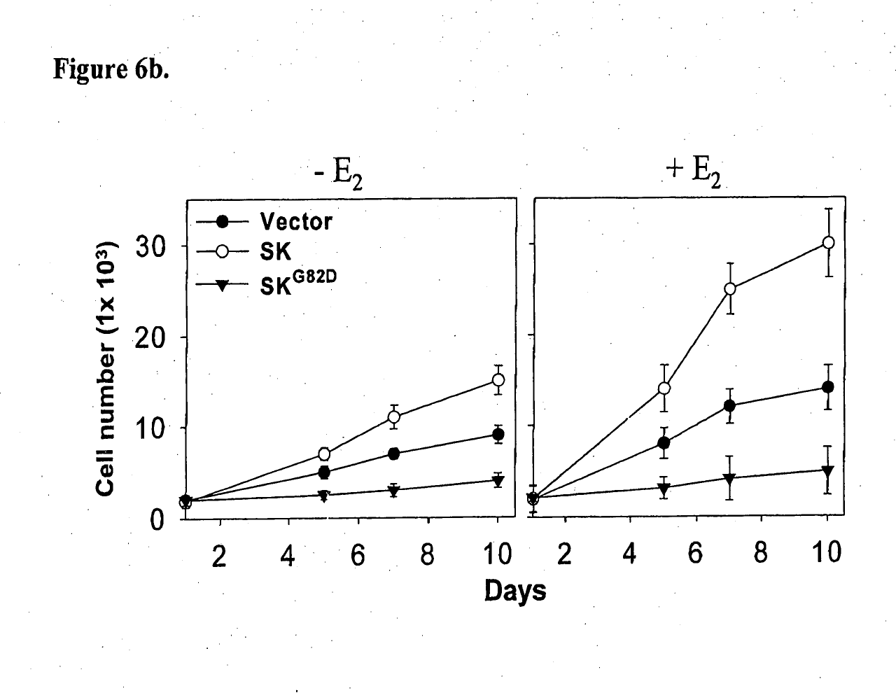

The ability of E₂ to promote fast intracellular Ca²⁺ mobilization has been demonstrated as a typical nongenomic action of E₂ through yet unknown transmembrane signal transduction pathway(s) (Hall, J. M., Couse, J. F., and Korach, K. S. (2001) J Biol Chem, 276: 36869-36872; Morley, P., Whitfield, J. F., Vanderhyden, B. C., Tsang, B. K., and Schwartz, J. L. (1992) Endocrinology, 131: 1305-1312). S1P, the product of SK activation, has been shown not only to induce intracellular Ca²⁺ mobilization by itself, but also serve as a second messenger to mediate Ca²⁺ mobilization in responses to a variety of stimuli, such as growth factors, cytokines and GPCR ligands (reviewed in Beaven, M. A. (1996) Curr. Biol, 6: 798-801; Young, K. W. and Nahorski, S. R. (2001) Semin. Cell Dev. Biol, 12: 19-25). Therefore, a role for SK activation in E₂-promoted intracellular Ca²⁺ mobilization was examined. At a physiological concentration of E₂ (1 nM) induced a rapid rise in intracellular free Ca²⁺ concentration ([Ca2+]ᵢ) that reached maximal level in 30±10 seconds as determined in Fluo-3 loaded MCF-7 cell by using Ca²⁺ fluorescent spectroscopy (FIG. 6) and assayed by Ca²⁺-imaging confocal microscopy in single cells (data not shown). The E₂-induced rise of [Ca²⁺]ᵢ was significantly enhanced in the cells overexpressing SK, whilst it was markedly declined in the cells transfected with SK^{G82D} or pretreated with DMS, a specific inhibitor of SK (FIGS. 6a and b), suggesting a critical role of SK in the E₂-dependent Ca²⁺ mobilization. Furthermore, addition of exogenous S1P to MCF-7 cells resulted in an increase of [Ca²⁺]ᵢ (FIG. 6b), which is similar to what has been observed in other cell types (Mattie, M., Brooker, G., and Spiegel, S. (1994) J Biol Chem, 269: 3181-3188; Choi, O. H., Kim, J. H., and Kinet, J. P. (1996) Nature, 380: 634-636; Meyer Zu, H. D., Lass, H., Alemany, R., Laser, K. T., Neumann, E., Zhang, C., Schmidt, M., Rauen, U., Jakobs, K. H., and van Koppen, C. J. (1998) EMBO J, 17: 2830-2837), supporting the role of SK activity in the signaling of Ca²⁺. In consistent with the ability of membrane impermeable E₂-BSA to activate SK, E₂-BSA induced increases in [Ca²⁺]ᵢ to same extent as E₂ did, whereas BSA alone was ineffective in affecting [Ca²⁺]ᵢ (FIG. 6c). The E₂-induced Ca²⁺ signaling was not prevented upon pre-incubation of the cells with nuclear ER blockers ICI 182,780 (FIG. 6d) or OH-Tam.

When extracellular Ca²⁺ was removed by 2 mM EGTA (FIG. 6e) or pretreated with inorganic Ca²⁺-channel blocker Ni²⁺, E₂ was still able to increase [Ca²⁺]ᵢ albeit the signals were slightly decreased, suggesting that the increase of [Ca2+]ᵢ resulted chiefly from intracellular Ca²⁺ stores. As expected, pretreatment with 100 nM of tert-butylhydroquinone (BHQ) (endoplasmic reticulum Ca²⁺-ATPase blockers) strongly reduced E₂-dependent increase in [Ca²⁺]ᵢ (FIG. 6e). Interestingly, a compound known to block IP₃ receptor (Soulsby, M. D. and Wojcikiewicz, R. J. (2002) Cell Calcium, 32: 175-181), 2-Aminoethoxydiphenyl borate (2-APB) was unable to effectively abolish the effects E₂ on [Ca²⁺]ᵢ (FIG. 4g,h,i). However, in the presence of DMS plus 2-APB, the E₂-promoted [Ca²⁺]₁ was almost completely abrogated (FIG. 6f), which supports previous finding that S1P can directly stimulate Ca²⁺ release from intracellular stores via a novel pathway that does not involve other mediators of Ca²⁺ release such as IP₃ or cADP ribose (Ghosh, T. K., Bian, J., and Gill, D. L. (1994) J Biol Chem, 269: 22628-22635).

SK Activity is Required for E₂-Dependent Erk Activation.

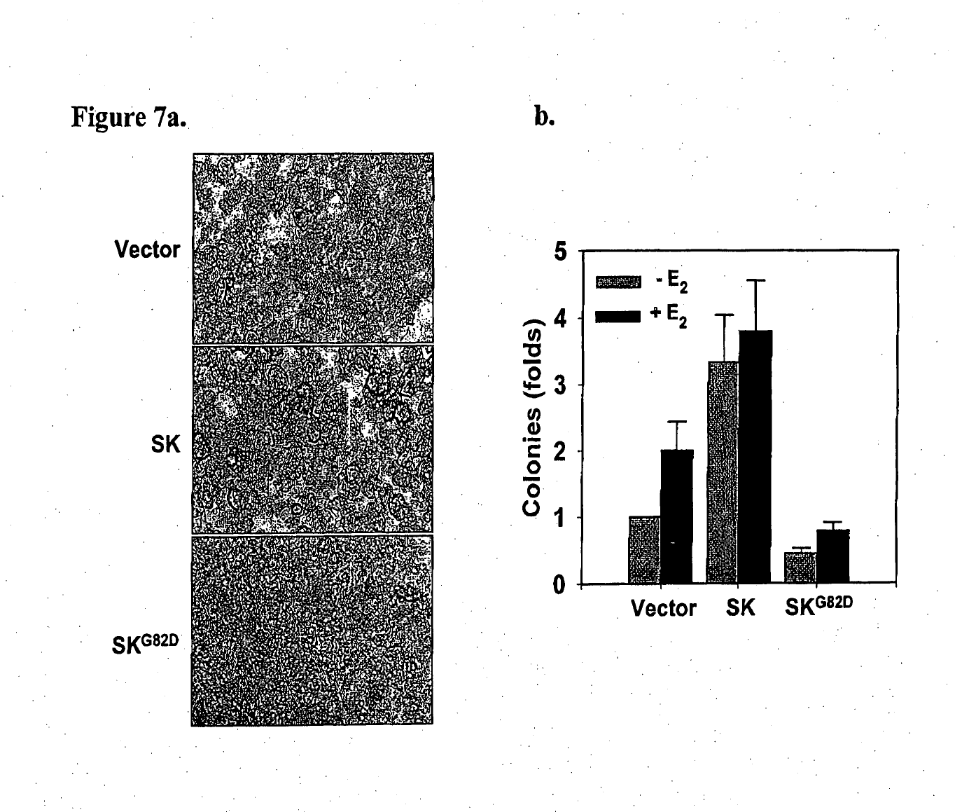

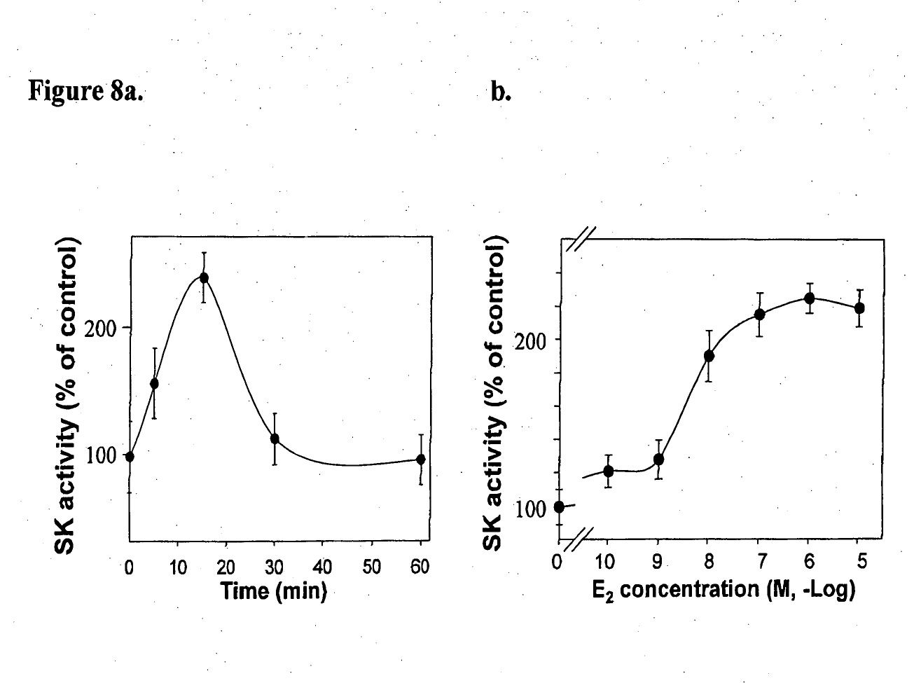

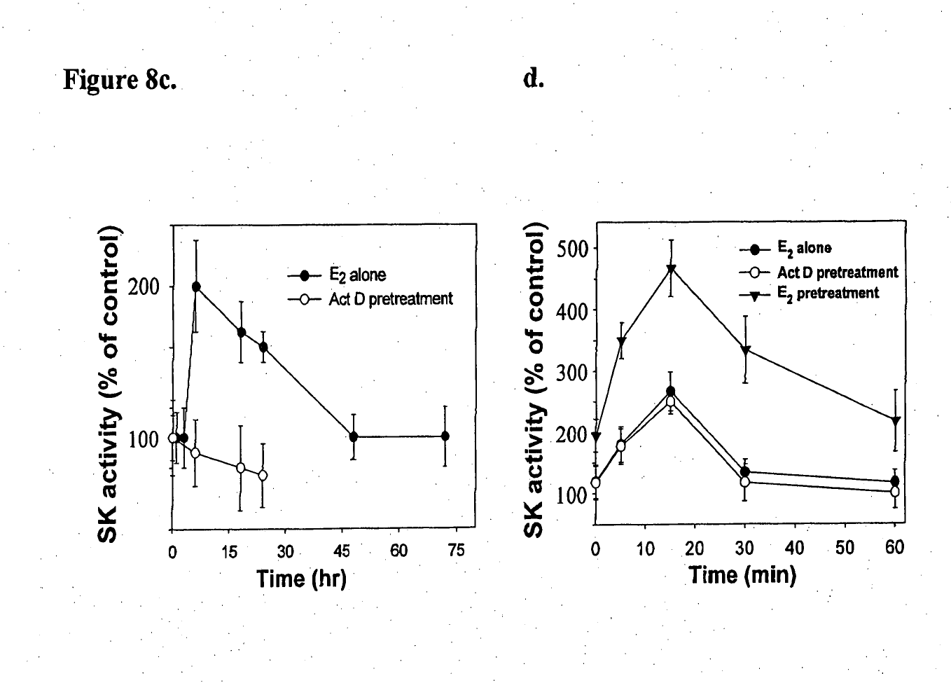

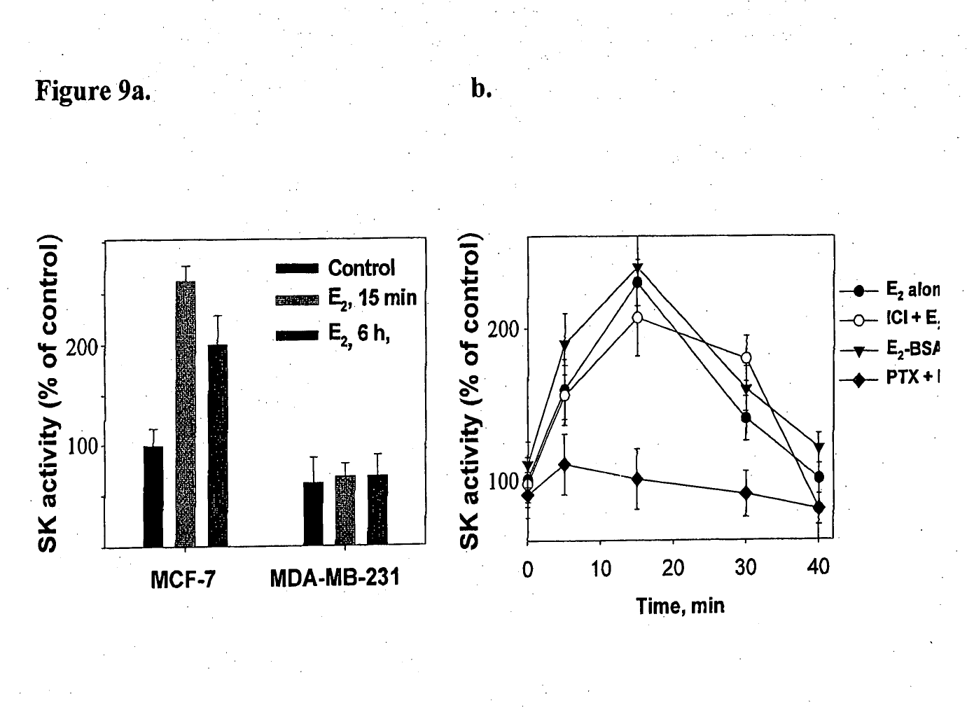

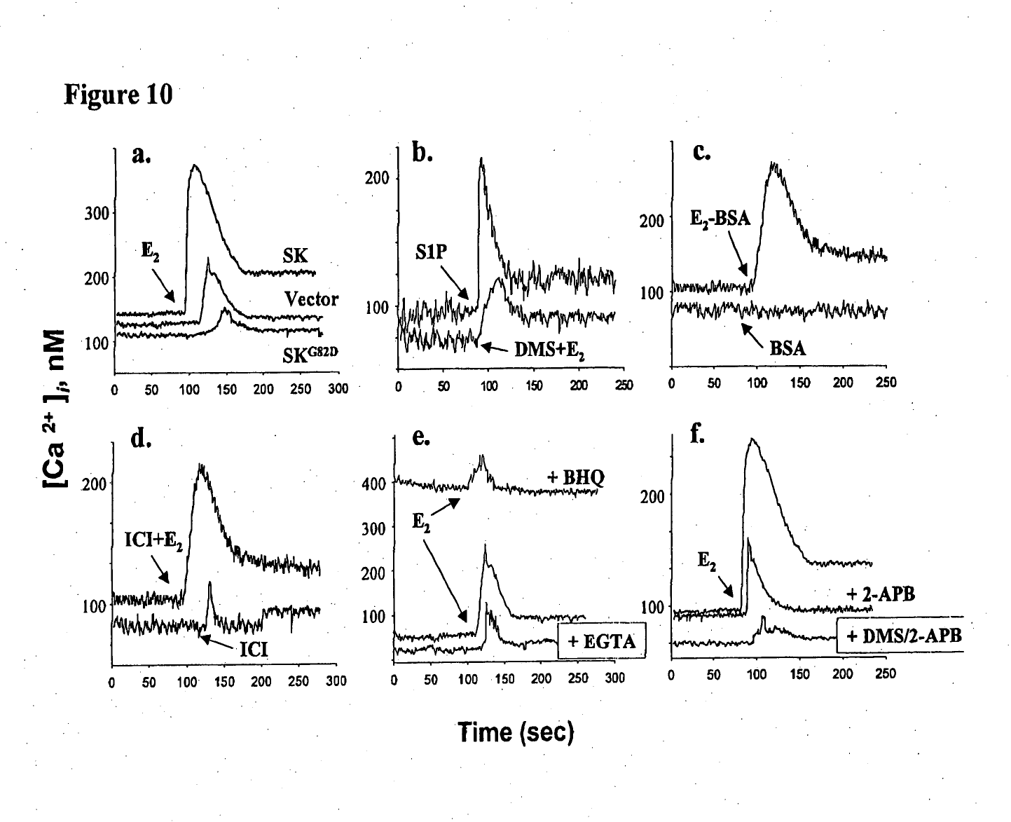

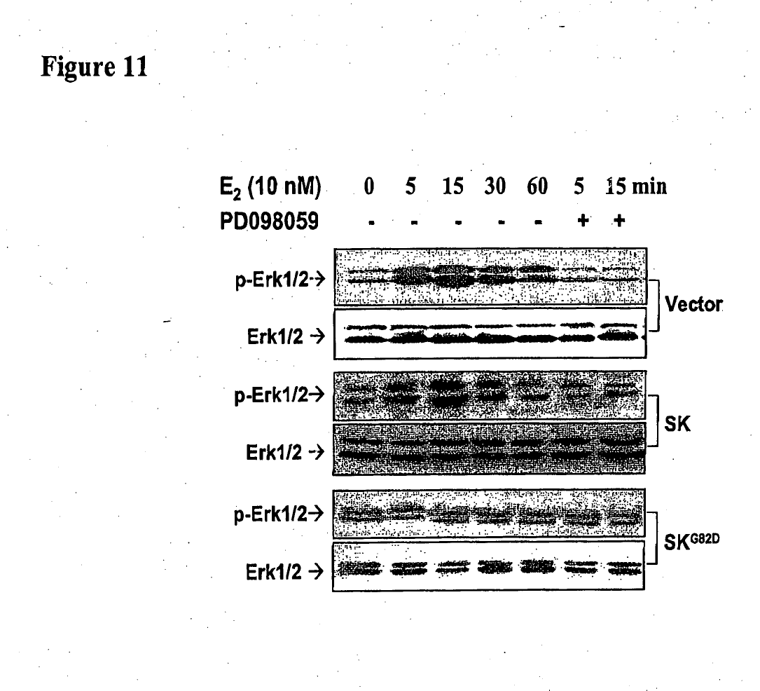

Erk activation has been well-documented as an important cytoplasmic signaling for E₂-mediated cell proliferation (Filardo, E. J., Quinn, J. A., Bland, K. I., and Frackelton, A. R., Jr. (2000) Mol Endocrinol., 14: 1649-1660; Migliaccio, A., Di Domenico, M., Castoria, G., De Falco, A., Bontempo, P., Nola, E., and Auricchio, F. (1996) EMBO J, 15: 1292-1300; Wade, C. B., Robinson, S., Shapiro, R. A., and Dorsa, D. M. (2001) Endocrinology, 142: 2336-2342). Previous work has revealed a role of SK in activation of Ras and Erk induced by cytokines and mitogenes (Xia et al., 2000, supra; Xia et al., 1998, supra). It was of interest to define whether SK activity is involved in E₂-induced Erk activation. Erk activity is tightly controlled by dual phosphorylation of residues Thr-183 and Tyr-185 of the proteins which is recognized on the immunoblot by using specific antibodies against the phosphorylated forms of Erk1/2. In agreement with previous reports (Filardo et al., 2000, supra; Migliaccio et al., 1996, supra), treatment of MCF-7 cells with E₂ resulted in a significant increase in Erk 1/2 activity with maximal activity being observed within 15 min treatment and returning to basal levels by 60 min (FIG. 7). Remarkably, the E₂-induced Erk1/2 phosphorylation was elevated in the wild type SK transfected MCF-7 cells, whereas the activation of Erk. was completely abrogated in the cells expressing dominant-negative SK (FIG. 7). As a control in these experiments, E₂-promoted phosphorylation of Erk1/2 was blocked by PD098095, a specific inhibitor of MEK, the upstream activator of Erk1/2 (FIG. 7). These data suggest that SK activation is an important signal to couple E₂ mediated Erk1/2 activation in MCF-7 breast cancer cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2
Transformation assays in transfected NIH 3T3 cells.

| | |
|---|---|
| Focus | |
| formation | Colonies in soft agar |

| Cell Line | Number | Number | Size (mm) |
|---|---|---|---|
| N-3T3 | 1.7 ± 1.5 | 3.3 ± 2.1 | <0.1 |
| SK-3T3 | 65.7 ± 9.6 | 122.3 ± 17.6 | 0.1˜0.45 |

Focux formation was assayed in NIH 3T3 cells transiently transfected with SphK (SK-3T3) or vector (N-3T3). Transfected cells were plated in 6-well plates and cultured for 2˜3 weeks prior to crystal violet staining. Colony formation in soft agar was determined in the stable transfected cells. Results shown are the mean ± SD from 3˜5 experiments done in duplicate or triplicate.

TABLE 3
Tumourigenesis in NOD/SCID mice.

| | |
|---|---|
| Tumours/ | Tumour size |

| Cell lines | injections | cm | cm³ |
|---|---|---|---|
| N-3T3 | 0/3 | 0 | |
| SK-3T3 | 3/3 | 2.8 × 1.8 × 1.1 | 5.54 |
| stable pools | | 1.9 × 1.5 × 1.0 | 2.85 |
| | | 1.1 × 0.9 × 0.8 | 0.79 |
| Clone KT-2 | 3/3 | 3.2 × 1.8. × 1.2 | 6.91 |
| | | 1.6 × 1.2 × 0.5 | 0.96 |
| | | 2.5 × 1.6 × 1.3 | 5.20 |
| Clone KT-5 | 3/3 | 2.7 × 1.6 × 1.2 | 5.18 |
| | | 2.2 × 1.5 × 1.1 | 3.63 |
| | | 1.8 × 1.7 × 0.9 | 2.75 |

NOD/SCID mice were injected with cells (5×10⁵ cells per mouse) from vector- or SphK-transfected NIH 3T3 cell pools (N-3T3 or SK-3T3), or two individual SphK-transfected clones (KT-2 and KT-5). Tumour size was determined 4 weeks after injection.

Bibliography

Allessenko, A. V., (1998) Biochemistry (Mosc) 63:62-68.
Beaven, M. A. (1996) Curr. Biol, 6: 798-801.
Benten, W. P., Lieberherr, M., Giese, G., and Wunderlich, F. (1998) FEBS Lett., 422: 349-353.
Benten, W. P., Stephan, C., Lieberherr, M., and Wunderlich, F. (2001) Endocrinology, 142: 1669-1677.
Choi, O. H., Kim, J. H., and Kinet, J. P. (1996) Nature, 380: 634-636.
Culliver, O., Pirianov, G., Kleuser, B., Vanek, P. G., Coso, O. A., Gutkind, J. S. and Spiegel, S. (1996) Nature 381:800-803.
Filardo, E. J., Quinn, J. A., Bland, K. I., and Frackelton, A. R., Jr. (2000) Mol Endocrinol., 14: 1649-1660.
Ghosh, T. K., Bian, J., and Gill, D. L. (1 994) J Biol Chem, 269: 22628-22635.
Gierthy, J. F., Lincoln, D. W., Roth, K. E., Bowser, S. S., Bennett, J. A., Bradley, L., and Dickerman, H. W. (1991) J Cell Biochem, 45: 177-187.
Grynkiewicz, G., Poenie, M., and Tsien, R. Y. (1985) J Biol Chem, 260: 3440-3450.
Guo, Z., Krucken, J., Benten, W. P., and Wunderlich, F. (2002) J Biol Chem, 277: 7044-7050.
Hall, J. M., Couse, J. F., and Korach, K. S. (2001) J Biol Chem, 276: 36869-36872.
Hyder, S. M., Chiappetta, C., Murthy, L., and Stancel, G. M. (1997) Cancer Res., 57: 2547-2549.
Igarashi, Y. (1997) J. Biochem. 122:1080-1087.
Kao, J. P., Harootunian, A. T., and Tsien, R. Y. Photochemically generated cytosolic calcium pulses and their detection by fluo-3. J Biol Chem, 264: 8179-8184, 1989
Kao, J. P., Harootunian, A. T., and Tsien, R. Y. (1989) J Biol Chem, 264: 8179-8184
Mattie, M., Brooker, G., and Spiegel, S. (1994) J Biol Chem, 269: 3181-3188.
Meyer zu Heringdorf, D., van Koppen, C. J. and Jakobs, K. H. (1997) FEBS Lett 410:34-38.
Meyer Zu, H. D., Lass, H., Alemany, R., Laser, K. T., Neumann, E., Zhang, C., Schmidt, M., Rauen, U., Jakobs, K. H., and van Koppen, C. J. (1998) EMBO J, 17: 2830-2837.
Migliaccio, A., Di Domenico, M., Castoria, G., De Falco, A., Bontempo, P., Nola, E., and Auricchio, F. (1996) EMBO J, 15: 1292-1300.
Morley, P., Whitfield, J. F., Vanderhyden, B. C., Tsang, B. K., and Schwartz, J. L. (1992) Endocrinology, 131: 1305-1312.
Musgrove, E. A. and Sutherland, R. L. (1994) Semin. Cancer Biol, 5: 381-389.
Pitson, S. M., Moretti, P. A., Zebol, J. R., Xia, P., Gamble, J. R., Vadas, M. A., D'andrea, R. J., and Wattenberg, B. W. (2000) J Biol Chem, 275: 33945-33950.
Pitson, S. M. et al. (2000) Biochem. J. submitted
Soulsby, M. D. and Wojcikiewicz, R. J. (2002) Cell Calcium, 32: 175-181.
Spiegel, S., Culliver, O., Edsall, L., Kohama, T., Menzeleev, R., Olivera, A., Thomas, D., Tu, Z., Van Brocklyn, J. and Wang, F. (1998) Biochemistry (Mosc) 63:69-73.
Wade, C. B., Robinson, S., Shapiro, R. A., and Dorsa, D. M. (2001) Endocrinology, 142: 2336-2342.
Xia, P., Gamble, J. R., Rye, K.-A., Wang, L., Hii, C. S. T., Cockerill, P., Khew-Goodall, Y., Bert, A. G., Barter, P. J., and Vadas, M. A. (1998) Proc. Natl. Acad. Sci. USA 95:14196-14201.
Xia, P., Vadas, M. A., Rye, K. A., Barter, P. J., Gamble, J. R. (1999) J. Biol. Chem. 274: 33143-33147.
Xia, P., Wang, L., Gamble, J. R., and Vadas, M. A. (1999) J Biol Chem, 274: 34499-34505.
Xia, P., Gamble, J. R., Wang, L., Pitson, S. M., Moretti, P. A., Wattenberg, B. W., D'andrea, R. J., and Vadas, M. A. (2000) Curr. Biol., 10: 1527-1530.
Young, K. W. and Nahorski, S. R.(2001) Semin. Cell Dev. Biol, 12: 19-25.

1. A method of modulating the growth of a cell, comprising contacting said cell with an effective amount of an agent for a time and under conditions sufficient to modulate the functional activity of sphingosine kinase wherein down-regulation of the functional activity of said sphingosine kinase down-regulates said growth and up-regulation of the functional activity of said sphingosine kinase up-regulates said cell growth.

2. A method of modulating the growth of a cell, comprising contacting said cell with an effective amount of an agent for a time and under conditions sufficient to modulate the level of functional activity of sphingosine kinase wherein down-regulation of the functional activity of said sphingosine kinase to an oncogenic ineffective level down-regulates said growth and up-regulation of the functional activity of said sphingosine kinase to an oncogenic effective level up-regulates said cell growth.

3. The method according to claim 2, wherein said growth is proliferation.

4. The method according to claim 3, wherein said modulation of proliferation is down-regulation of proliferation and said modulation of functional activity is down-regulation of functional activity.

5. The method according to claim 3, wherein said modulation of proliferation is up-regulation of proliferation and said modulation of functional activity is up-regulation of functional activity.

6. The method according to claim 4, wherein said proliferation is uncontrolled proliferation.

7. The method according to claim 6, wherein said cell is a neoplastic cell.

8. The method according to claim 7, wherein said neoplastic cell is a malignant cell.

9. The method according to claim 8, wherein said malignant cell is a cell from the colon, stomach, lung, brain, bone, esophagus, pancreas, breast, ovary or uterus.

10. The method according to claim 9, wherein said malignant cell is a breast cell.

11. The method according to claim 9, wherein said malignant cell has become transfected due to up-regulation of an oncogene.

12. The method according to claim 11, wherein said oncogene is Ras.

13. The method according to claim 9, wherein said malignant cell has become transformed by sphingosine kinase overexpression oncogenic activity.

14. The method according to any one of claims 1-4 or 6-13, wherein said agent is N,N-dimethylsphingosine.

15. The method according to any one of claims 1-4 or 6-13, wherein said agent is DL-threo-dihydrosphingosine.

16. A method for the treatment or prophylaxis of a condition characterized by aberrant, unwanted or otherwise inappropriate cell growth in a mammal, comprising administering to said mammal an effective amount of an agent for a time and under conditions sufficient to modulate the functional activity of sphingosine kinase.

17. A method for the treatment or prophylaxis of a condition characterized by aberrant, unwanted or otherwise inappropriate cell growth in a mammal, comprising administering to said mammal an effective amount of an agent for a time and under conditions sufficient to modulate the level of functional activity of sphingosine kinase wherein down-regulation of the functional activity of said sphingosine kinase to an oncogenic ineffective level down-regulates said growth and up-regulation of the functional activity of said sphingosine kinase to an oncogenic effective level up-regulates said cell growth.

18. The method according to claim 17, wherein said growth is proliferation.

19. The method according to claim 18, wherein said modulation of proliferation is down-regulation of proliferation and said modulation of functional activity is down-regulation of functional activity.

20. The method according to claim 18, wherein said modulation of proliferation is up-regulation of proliferation and said modulation of functional activity is up-regulation of functional activity

21. The method according to claim 19, wherein said proliferation is uncontrolled proliferation.

22. The method according to claim 21, wherein said cell is a neoplastic cell.

23. The method according to claim 22, wherein said neoplastic cell is a malignant cell.

24. The method according to claim 23, wherein said malignant cell forms a solid tumour tumor of the colon, stomach, lung, brain, bone, breast, esophagus or pancreas.

25. The method according to claim 23, wherein said malignant cell forms a solid tumour tumor of the breast.

26. The method according to claim 24, wherein said malignant cell has become transformed due to oncogene up-regulation.

27. The method according to claim 26, wherein said oncogene is Ras.

28. The method according to claim 24, wherein said malignant cell has become transformed by sphingosine kinase over expression oncogenic activity.

29. The method according to any one of claims 16-19 or 21-28, wherein said agent is N,N-dimethylsphingosine.

30. The method according to any one of claims 16-19 or 21-28, wherein said agent is DL-threo-dihydrosphingosine.

31. The method according to any one of claims 16-28, wherein said mammal is a human.

32. A pharmaceutical composition comprising an agent capable of modulating the functional activity of sphingosine kinase together with one or more pharmaceutically acceptable carriers and/or diluents for use in accordance with the method of any one of claims 1-13 or 16-28.

33. The pharmaceutical composition according to claim 32, wherein said agent is N,N-dimethylsphingosine.

34. The pharmaceutical composition according to claim 32, wherein said agent is DL-threo-dihydrosphingosine.

35. A method of diagnosing a condition, or a predisposition or resistance to a condition characterized by aberrant, unwanted or otherwise inappropriate cell growth in a mammal, said method comprising screening a biological sample from said mammal for the presence of sphingosine kinase or a nucleic acid molecule encoding sphingosine kinase.

36. The method according to claim 29, wherein said mammal is a human.

37. The method according to claim 30, wherein said mammal is a human.
